(19) United States
(12) Patent Application Publication
(10) Pub. No.: US 2013/0133108 A1
(43) Pub. Date: May 23, 2013

(54) EXTRACTS FROM PIRIN+ AND PIRIN- PLANTS AND USES THEREOF

(71) Applicants: Katherine Warpeha, Chicago, IL (US); Lon Kaufman, Highland Park, IL (US)

(72) Inventors: Katherine Warpeha, Chicago, IL (US); Lon Kaufman, Highland Park, IL (US)

(21) Appl. No.: 13/812,917

(22) PCT Filed: Aug. 25, 2011
(86) PCT No.: PCT/US2011/049220

§ 371 Date: Jan. 29, 2013

Related U.S. Application Data
(60) Provisional application No. 61/376,914, filed on Aug. 25, 2010.

(51) Int. Cl. C12N 15/82

(52) U.S. Cl. CPC C12N 15/8257
USPC 800/279; 549/400; 800/278; 800/289; 514/456; 504/292; 424/725

(57) ABSTRACT

The present disclosure provides methods of making and using plant extracts that include quercetin, which are generated from transgenic plants that have decreased pirin activity (prn-). Such transgenic plants and their extracts can be used to increase tolerance of a plant to a stressor, such as UV light, as well as treat tumor cells (such as kill cancer cells), prevent certain types of fungal infections (such as C. gattii), and increase antioxidant activity. The present disclosure also provides methods of making and using plant extracts that are depleted of quercetin, which are generated from transgenic plants that have increased pirin activity (prn+). Such transgenic plants and their extracts can be used to prevent certain types of fungal infections (such as C. neoformans). Also provided are compositions that include the prn- or prn+ extracts, such as a plastic coated with the extract.

### CROSS REFERENCE TO RELATED APPLICATION

This application claims priority to U.S. Provisional Application No. 61/376,914 filed Aug. 25, 2010, herein incorporated by reference.

### FIELD

The present disclosure provides methods of making and using plant extracts generated from transgenic plants that have decreased pirin activity (prn-) or increased pirin activity (prn+), such as pirin1 activity. Also provided are compositions that include the prn− or prn+ extracts, such as a plastic coated with the extract.

### BACKGROUND

Quercetin is the most abundant molecule in the large class of polyphenolic flavonoids and synthesized by most if not all plants. The reported bioactivities of quercetin include antioxidative, antiviral, antibacterial and anti-inflammatory effects. Pirin (abbreviated PIR or PRN) is an iron-containing protein that is a focus of interest in apoptosis and cellular stresses, particularly that related to malignancy (Licciulli et al., Leukemia. 24(2):429-37, 2010; Miyazaki et al., Nat Chem. Biol. 6(9):667-73, 2010; Yoshikawa et al., Oncol Rep. 12(6):1287-93, 2004; Hihara et al., FEBS Lett. 574(1-3):101-5, 2004). Pirin protein was first identified as an interactor of nuclear factor I/CCAAT box transcription factor NFI/CTF1 to drive adenovirus DNA replication and polymerase II transcription (Wendler et al., J. Biol. Chem. 272(13):8482-9, 1997). Prn is highly conserved between prokaryotes, fungi, plants and mammals and is weakly expressed in many human tissues (Id.). Due to sequence and structural similarity, Prn has been classified as a member of the functionally diverse cupin superfamily (Id. and Dunwell, Phytochemistry 65:7-17, 2004). In eukaryotes, Prn has been implicated as having possible involvement in a wide variety of biological processes, such as apoptosis (Orzaez et al, 2001), germination in Arabidopsis (Lapik and Kaufman, Plant Cell. 15(7):1578-90, 2003), transcriptional regulation (Chen et al., Annu. Rev. Genet. 38:87-117, 2004), stress responses (Hihara et al., FEBS Lett. 574(1-3):101-5, 2004) and in regulation of genes with CCAAT-box binding regions in the 5′UTR (Warpeha et al., Plant Physiol. 144(4):1590-1600, 2007). More recently, Prn was found to possess enzymatic activity, with roles as a quercetinase in both bacteria and humans where quercetin is cleaved resulting in carbon monoxide and 2-protocatechuoylphloroglucinol carboxylic acid (Adams and Jai, J. Biol. Chem. 280(31):28675-82, 2005; Oka and Simpson, Biochem Biophys Res Commun. 43(1):1-5, 1971). Other than its antioxidant properties in animals that ingest it, quercetin is an important flavonoid for plants cells in that it can absorb ultraviolet radiation as a natural sunscreen, preventing damage by UV radiation (REF).

### SUMMARY

It is shown herein that Prn functions as a regulatory “switch” in the daily cycle of the young plant. A heterotrimeric G protein pathway (putative G-protein-coupled receptor 1 [GCR1], and G-protein α subunit 1 [GPA1]) in the genetic model Arabidopsis thaliana interacts with Prn to elicit its activities in developing leaf cells of very young plants. In dark-grown seedlings less than 7 days old only one pirin is expressed, AtPirin1. In absence of an abiotic stimulus (no light, room temperature, no external stressors), AtPirin1 can break down the levels of quercetin, a potent antioxidant. When the G-protein is activated (situation of transcriptional activation), quercetinase activity is not measured. In presence of light (day) AtPirin1 acts as a transcriptional regulator for abiotically-regulated genes. Thus, Prn can regulate quercetin levels, a metabolite with a function in cellular defense systems. It is also shown herein that prn- knock-out plants are protected from stressors, and that extracts from such plants can kill cancer cells and prevent infection by toxic fungi such as C. gattii. Based on these observations, methods of making and using prn- extracts, and prn+ extracts, are provided.

The present disclosure provides methods of making plant extracts (such as a seedling extract) that include quercetin. In particular examples, the method includes extracting or homogenizing the aerial portions (cotyledons or cotyledons and stem) of seedlings from a transgenic plant, wherein the transgenic plant includes an exogenous nucleic acid molecule that decreases or eliminates pirin expression or activity in the transgenic plant (such as a t-DNA insertion), thereby increasing an amount of quercetin in the transgenic plant. In some examples, such a transgenic plant has increased levels of quercetin (such as an increase of at least 2-fold, at least 3-fold, or at least 4-fold), for example relative to a comparable non-transgenic plant. A supernatant is obtained from the resulting prn- homogenate, thereby generating an extract that includes quercein. The resulting extract is referred to herein as a prn- extract. prn- extracts made by the disclosed methods are also provided.

In some examples, the method of making the prn- extract includes planting seeds of the transgenic plant in darkness in the cold, for example at 0° C. to 5° C. for 24 to 72 hours. The seeds of the transgenic plant are then grown in darkness at room temperature, such as 15° C. to 20° C. for 5 to 7 days. The transgenic plant can then exposed to one or more stressors, thereby generating an exposed transgenic plant (which can have additional metabolites), which is grown in darkness at room temperature, such as at 15° C. to 20° C. for 6 to 36 hours. Subsequently, cotyledons or the aerial portions of seedlings from the exposed transgenic plant are obtained and homogenized to generate a prn- extract. In some examples, as an alternative to making the prn− extract from the aerial portions of seedlings, the extract is made from prn− plant cells grown in culture, and treated as described above.

The disclosure also provided methods of making quercetin from plants. In particular examples the methods include extracting aerial portions of seedlings from a transgenic plant seedling having an exogenous nucleic acid molecule (such as a t-DNA insertion) that decreases or eliminates prn expression or activity in the transgenic plant, thereby decreasing or even eliminating functional prn protein in the transgenic plant, and increasing quercetin in the plant. A supernatant from the prn- homogenate is obtained, thereby generating a prn- extract that includes one or more quercetins, and other components, such as one or more of a propionic acid derivative, carbonic anhydrase, piperidine naphthalene-2-carboximida derivative, a benzoic acid derivative, inhibitor of glyoxalase, theanine derivative, di(n-acetyl-d-glucosamine, prednicarbate, coelenterazine-like compound, pyrrolo-pyrazole derivative, sulfonamide inhibitor of carbonic acid, methylsalicyluric acid, a compound, highly similar to 2S-hydroxy-10-undecanoic acid, a compound similar to aminobenzofurazan, antibiotics, a carbonic anhydrase inhibitor. The prn- extract can then be treated to isolate the quercetin. Also provided is isolated quercetin made by the disclosed methods.

Also provided are methods of increasing the tolerance of a plant to a stressor, such as UV light, salt or heat. The method can include expressing in the plant (such as a young plant) an exogenous nucleic acid molecule (such as a t-DNA insertion) that decreases or eliminates pirin expression or activity in the plant, thereby decreasing or eliminating functional pirin protein in the plant and increasing tolerance of the resulting transgenic plant to the stressor. In some examples, such a transgenic plant has increased levels of quercetin (such as an increase of at least 2-fold, at least 3-fold, or at least 4-fold), for example relative to a comparable non-transgenic plant. In some examples, the plant in need of increased stress tolerance is selected, such as one grown under stressor conditions. For example, the plant can be a highly inbred crop, such as rice, soybean, corn, cotton, wheat, oats, barley, or sorghum, which is likely to be exposed to one or more stressors during the growing season, such as chilling (cold), heat, salt, high light/UV, flooding, drought/water stress, or predators such as insects, parasitic worms, or arachnids.

Methods of using the prn- extracts are also provided. For example, the prn- extracts can be used to increase tolerance of a plant to a stressor. For example, by exposing or contacting the plant with the prn- extract (such as apply or spraying the extract to the outside of the plant, or growing the plant in soil or water containing the prn- extract), this can increase an amount of quercetin in the plant and increase tolerance of the plant to the stressor.

In another example, the prn- extracts are used to treat a tumor cell, for example kill a tumor cell. For example, the tumor cell to be treated can be contacted with a therapeutically effective amount of the prn- extract in vivo, ex vivo, or in vitro, thereby treating the tumor cell (for example by reducing growth of the tumor cell or killing the tumor cell).

In another example, the prn- extracts are used to increase anti-oxidant activity in a subject. For example, the method can include administering to a mammal a therapeutically effective amount of the prn- extract, thereby increasing anti-oxidant activity in the subject.

In another example, prn- extracts are used to reduce or prevent an infection in a mammalian subject or plant by toxic fungus, such as C. gattii. For example, the mammalian subject or plant can be contacted with a therapeutically effective amount of the prn- extract, thereby reducing or preventing infection of the mammal or plant by a toxic fungus. In another example, prn- extracts can be used to prevent a toxic fungus from growing on a surface (or significantly reduce its ability to do so). For example, the method can include contacting a surface with a therapeutically effective amount of the prn1 extract, thereby significantly reducing or preventing the ability of the fungus from growing on the surface.

The present disclosure also provides methods of making plant extracts that depleted or even lacking quercetin. In particular examples, the method includes extracting or homogenizing aerial portions of seedlings from a transgenic plant seedling, wherein the transgenic plant includes an exogenous nucleic acid molecule that increases pirin activity in the transgenic plant, thereby decreasing an amount of quercetin in the transgenic plant and extracts made from the plant. The resulting extract is referred to herein as a prn+ extract. Also provided are prn+ extracts made using this method.

In some examples, the method of making the prn+ extract includes planting seeds of the prn+ transgenic plant in darkness in the cold, for example at 0° C. to 5° C. for 24 to 72 hours. The seeds of the transgenic plant are then grown in darkness at room temperature, such as 15° C. to 20° C. for 7 days or less, such as 5 to 7 days, such as 6 to 7 days. Subsequently, the aerial portions of seedlings from the exposed transgenic plant seedling are obtained and homogenized to generate a prn+ extract. In some examples, as an alternative to making the prn+ extract from the aerial portions of seedlings, the extract is made from prn+ plant cells grown in culture, and treated as described above.

In one example, the prn+ extracts are used to reduce or prevent an infection in a mammalian subject or plant by a fungus that requires quercetin, or a fungus that has laccase activity, such as C. neoformans. For example, the mammalian subject or plant can be contacted with a therapeutically effective amount of the prn+ extract, thereby reducing or preventing infection of the mammal or plant by a fungus that requires quercetin, or a fungus that has laccase activity. In another example, the prn+ extracts are used to prevent a fungus that requires quercetin, or a fungus that has laccase activity from growing on a surface (or significantly reduce its ability to do so). For example, the method can include contacting a surface with a therapeutically effective amount of the prn+ extract, thereby significantly reducing or preventing the ability of the fungus from growing on the surface.

Extracts made by the disclosed methods are also provided. Such extracts can further include other components, such as a pharmaceutically acceptable carrier. Also provided are compositions that include the disclosed extracts. For example, compositions that include a plastic material and a prn- or prn+ extract on a surface of the plastic material, are provided

The foregoing and other objects and features of the disclosure will become more apparent from the following detailed description, which proceeds with reference to the accompanying figures.

### BRIEF DESCRIPTION OF THE DRAWINGS

FIG. 1 is a graph showing that PRN1 specifically cleaves quercetin; and that quercetinase activity is not due to the in vitro translation protein extract itself which contains all components of translation and assay except PRN1.

FIG. 2 is a graph showing that PRN1 quercetinase activity is regulated through its interactions with GPA1, turning off as a result of interaction with activated GPA1 (non-hydrolyzable GTPγS=always ON conformation), but active and cleaving quercetin when GPA1-GDPα=always OFF conformation.

FIG. 3 is a bar graph showing that elimination of the PRN1 gene leads to high levels of quercetin, but not closely related compounds such as kaempferol, in etiolated seedlings.

FIGS. 4A and 4B are digital images showing features of the prn1 mutant seedling. (A) Seedlings were grown for 7 days in complete darkness, on day 6 seedlings were treated with a large dose of UV-C radiation: 10⁵ mM m⁻² UVC radiation (wild type can survive 10⁴). While wild type did not survive and pd1/adt3 mutants die from very low doses of UV-C, prn1 mutants survive. (B) The natural fluorescence of mutants involved in G-protein signaling (Warpeha et al., Plant Physiol. 140:844-55, 2006), and unlike other components of the pathway, prn1 mutants make an excess of pigments/light absorbing compounds. BL=blue light treatment on day 6; harvest day 7 as shown.

FIG. 5A is a model showing the events in the absence of abiotic signal or pre-stress. In etiolated Arabidopsis seedlings, only low levels of Phe and quercetin are synthesized and produced. PRN1 acts as a quercetinase, actively degrading any quercetin made.

FIG. 5B is a model showing the events in the presence of abiotic signal or pre-stress. Activation of the GCR1-GPA1 pathway via an abiotic signal (e.g., UV-B, heat, salt) or pre-stress activators (e.g., BL, UV-A) leads to the activation of PD1/ADT3, enhanced Phe synthesis, and thereby enhanced quercetin synthesis. Simultaneously, PRN1 quercetinase activity is switched off, allowing PRN1 to interact with NFY to carry out its role as a transcriptional activator.

FIGS. 6A and 6B are graphs showing growth of (A) MCF-7 cells or (B) MCF-10a cells in the presence of extracts from wild-type Arabidopsis (WT), pd1 mutants (PD1), prn1 mutants (PRN), or a mixture of all three (Mix). Cell growth was normalized to normal cells or non-invasive untreated cancer cells.

FIGS. 7A and 7B show a pirin subdomain is significantly conserved between mammals, plants, and prokaryotes. Reproduced from Wendler et al. (J. Biol. Chem. 272:8482-9, 1997). (A) The N-terminus of human Pirin (amino acids 1-136 SEQ ID NO: 4) contains 29 amino acids between Gly52 and Tyr131 that are highly conserved throughout all aligned sequences. These are derived from mouse (Mou, amino acids 87-136 of SEQ ID NO: 5), A. thaliana (Ara, amino acids 21-109 of SEQ ID NO: 2), D. discoideum (Dic, Z29535; amino acids 8 to 101 of SEQ ID NO: 6), A. acidocaldarius (Bac, amino acids 32 to 126 of SEQ ID NO: 7), S. coelicolor (Str, amino acids 16 to 93 of SEQ ID NO: 8), and E. coli (Eco, amino acids 1 to 135 of SEQ ID NO: 9). (B) the C terminus of human Pirin (amino acids 137-236 SEQ ID NO: 4) aligned against mouse EST WO8720 [GenBank]; amino acids 137 to 230 of SEQ ID NO: 5, rat EST AA012706; amino acids 166 to 226 of SEQ ID NO: 10, and the hypothetical protein from E. coli (P46852[GenBank]; amino acids 136 to 224 of SEQ ID NO: 9). Identical or similar residues conserved among all sequences are shown in black, and residues not fully conserved are marked by gray boxes.

FIG. 8 shows an alignment of pirin sequences from E. coli (Ec) (SEQ ID NO: 9), Homo sapiens (Hs) (amino acids 1-287 of SEQ ID NO: 4) and A. thaliana (At) (SEQ ID NO: 2).

FIGS. 9A and 9B are graphs showing that co-incubation of seeds of Arabidopsis thaliana with fungal cells of C. neoformans results in seedling death. Seeds of either wt (wtAt), Atpd1/adt3Δ (pd1) or Atprn1Δ (prn1) mutants were incubated with 1×10⁶ CFU of wt strain H99 of C. neoformans and grown for the first 48 h in the dark, then moved to either by dim light (panel A) or bright light (panel B). Seeds that never germinated or died in germination as determined by microscopy were scored as not surviving at day 1 (germination) and seedlings were monitored for stem lodging at day 14 and day 21. P values between indicated groups are displayed.

FIGS. 10A and 10B are graphs showing that co-incubation of seeds of Arabidopsis thaliana with fungal cells of C. gattii results in seedling death. Indicated seeds were co-incubated with wt strains of C. gattii as in FIGS. 9A and 9B. P values between indicated groups are displayed.

FIG. 11 is a bar graph showing that co-incubation of seeds of A. thaliana with fungal cells of Cryptococcus result in significant cryptococcal fungal burdens. Indicated seeds were co-incubated with indicated fungal strains as in FIG. 9 in dim light. At 14 days, intact seedlings were recovered, separated from root material, washed extensively, homogenized and fungal CFU burden determined per gram of plant tissue.

FIG. 12 provides digital images showing that co-incubation of A. thaliana with C. neoformans or C. gattii results in fungal tissue invasion of plant tissue. Indicated seeds and fungal cells were co-incubated in dim light as in FIG. 9. At 14 days, seedlings were harvested, fixed and subjected to tissue embedding and sectioning, followed by staining with either Gomori-silver (left panels) or hematoxylin-eosin (right panels). Arrows show fungal cells.

FIG. 13 are digital images and a graph showing the role of laccase in plant pathogenicity of C. neoformans. Plants were inoculated as in FIG. 9 with the indicated strains and then at 21 days, observed for morphological changes and stem lodging/death. P values between indicated groups are shown.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. All sequence database accession numbers referenced herein are understood to refer to the version of the sequence identified by that accession number as it was available on the filing date of this application. In the accompanying sequence listing:

SEQ ID NOS: 1 and 2 provide exemplary Arabidopsis thaliana pirin1 nucleic acid and protein sequences, respectively.

SEQ ID NOS: 3 and 4 provide exemplary human pirin nucleic acid and protein sequences, respectively.

SEQ ID NO: 5 provides an exemplary mouse pirin protein sequence.

SEQ ID NO: 6 provides an exemplary D. discoideum pirin protein sequence.

SEQ ID NO: 7 provides an exemplary A. acidocaldarius pirin protein sequence.

SEQ ID NO: 8 provides an exemplary S. coelicolor pirin protein sequence.

SEQ ID NO: 9 provides an exemplary E. coli pirin protein sequence.

SEQ ID NO: 10 provides an exemplary rat pirin protein sequence.

SEQ ID NOS: 11 and 12 provide exemplary Zea mays pirin nucleic acid and protein sequences, respectively.

SEQ ID NOS: 13 and 14 provide exemplary Ricinus communis pirin nucleic acid and protein sequences, respectively.

SEQ ID NOS: 15 and 16 provide exemplary Carica papaya pirin nucleic acid and protein sequences, respectively.

### DETAILED DESCRIPTION

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a plant cell” includes a plurality of such cells and reference to “the vector” includes reference to one or more vectors and equivalents thereof known to those skilled in the art, and so forth. Similarly, the word “or” is intended to include “and” unless the context clearly indicates otherwise. Hence “comprising A or B” means including A, or B, or A and B.

Unless explained otherwise, all technical and scientific terms used herein have the same meaning as commonly understood to one of ordinary skill in the art to which this disclosure belongs.

All references and GenBank accession numbers are incorporated by reference. The sequences present on Aug. 25, 2011 in the GenBank accession numbers are incorporated by reference.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, the cell killed by the disclosed methods is a cancer cell.

Contacting: Placement in direct physical association, including both a solid and liquid form. Contacting can occur in vitro, for example, with isolated cells, such as plant cells, or in vivo by administering to a subject (such as a subject with a tumor).

Control: A sample or standard used for comparison, for example for comparison to a non-native activity. In some embodiments, a wild-type (wt) plant (e.g., seedling) or plant cell serves as a control for a transgenic plant (e.g., seedling) or plant cell (e.g., a prnprn− or prn+ transgenic plant or seedling). In some embodiments, the control is an untreated sample or subject, such as a subject or sample not contacted or exposed to a prn− or prn+ extract. In some embodiments, the control is a historical control or standard value (i.e. a previously tested control sample or group of samples that represent baseline or normal values). In some embodiments the control is a standard value representing the average value (or average range of values) obtained from a plurality of samples. For example, the control can be a historical or standard value or range of values representing quercetin or Prn activity expected in a wt plant. In another example, the control can be a historical or standard value or range of values representing tumor cell or tumor killing activity expected by a prn− extract or by no treatment.

Decrease: To reduce the quality, amount, or strength of something.

In one example, a prn- plant or seedling has decreased or eliminated (e.g., non-detectable) prn expression or prn activity, for example as compared to prn expression or prn activity in a wt plant or seedling. In some examples, the decrease in to prn expression or prn activity is at least 20%, at least 50%, at least 75%, at least 90%, at least 100%, or even at least 200%, relative to the amount observed with a wt plant or seedling.

In one example, a therapeutic composition that includes prn− extract decreases the viability of tumor cells, for example as compared to the response in the absence of the prn− extract. In some examples such a decrease is evidenced by increased killing of the tumor or tumor cells, decreased tumor growth, decreased tumor size, decreased tumor volume, and the like. In some examples, the decrease in the viability of tumor cells, size of tumor, volume of tumor, or rate of growth is at least 20%, at least 50%, at least 75%, at least 90%, at least 100%, or even at least 200%, relative to that observed with a composition that does not include a prn− extract.

In another example, a prn+ plant or seedling has decreased production of quercetin, for example as compared to the quercetin produced in a prn− plant or seedling. In some examples, the decrease in quercetin is at least 20%, at least 50%, at least 75%, at least 90%, at least 100%, or even at least 200%, relative to the amount observed with a prn− plant or seedling.

In other examples, decreases are expressed as a fold change, such as an decrease in prn expression or prn activity; tumor cell viability, growth, volume or size; or an amount of quercetin by at least 2-fold, at least 3-fold, at least 4-fold, at least 5-fold, at least 8-fold, at least 10-fold, or even at least 15 or 20-fold, relative to the appropriate control (e.g., wt plant/seedling or untreated tumor). Such decreases can be measured using the methods disclosed herein.

Degenerate variant: A polynucleotide encoding a peptide, such as a Prn peptide, that includes a sequence that is degenerate as a result of the genetic code. There are 20 natural amino acids, most of which are specified by more than one codon. Therefore, all degenerate nucleotide sequences are included as long as the amino acid sequence of the peptide encoded by the nucleotide sequence is unchanged.

Down-regulated or inactivation: When used in reference to the expression of a nucleic acid molecule, such as a gene, refers to any process which results in a decrease in production of a gene product, such as Prn. A gene product can be RNA (such as mRNA, rRNA, tRNA, and structural RNA) or protein. Therefore, gene down-regulation or deactivation includes processes that decrease transcription of a gene or translation of mRNA.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene down-regulation can include reduction of expression above an existing level. Examples of processes that decrease translation include those that decrease translational initiation, those that decrease translational elongation and those that decrease mRNA stability.

Gene down-regulation includes any detectable decrease in the production of a gene product. In certain examples, production of a gene product decreases by at least 2-fold, for example at least 3-fold or at least 4-fold, as compared to a control (such an amount of gene expression in a non-transgenic seedling cell). In one example, a control is a relative amount of gene expression in a corresponding non-transgenic plant or seedling of the same variety of the transgenic plant or seedling.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule can be modulated compared to a normal (wild type) nucleic acid molecule. Modulation includes but is not limited to: (1) overexpression; (2) underexpression; or (3) suppression of expression. Modulation of the expression of a nucleic acid molecule can be associated with, and in fact cause, a modulation in expression of the corresponding protein.

Exogenous: The term “exogenous” as used herein with reference to nucleic acid and a particular cell refers to any nucleic acid that does not originate from that particular cell as found in nature. Thus, a non-naturally-occurring nucleic acid is considered to be exogenous to a cell once introduced into the cell. A nucleic acid that is naturally-occurring also can be exogenous to a particular cell. For example, a pirin encoding sequence from a cell or recombinantly produced s an exogenous nucleic acid with respect to another cell once that pirin encoding sequence is introduced into the other cell.

Functional deletion (gene inactivation): A mutation, partial or complete deletion, insertion, or other variation made to a gene sequence which significantly reduces or even inhibits production of the gene product, and/or renders the gene product non-functional. Also referred to as a mutation that inactivates the gene. In some examples, inactivation of pirin in a plant seedling decreases pirin activity in the seedling by at least 20%, at least 50%, at least 75%, at least 90%, at least 95%, or even at least 100%, relative to the activity observed with a seedling that includes functional pirin. For example, functional deletion of pirin in a plant seedling increases the production of quercetin in the seedling (for example an increase of at least 2-fold, at least 3-fold, or at least 4-fold, relative to the amount of quercetin observed with a seedling that includes functional pirin), as pirin is a quercetinase. This functional deletion of PRN (such as PRN1) in seedlings increases the ability of the seedling (such as a young plant) to tolerate exposure to stressors.

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (detects sequences that share at least 90% identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (detects sequences that share at least 80% identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (detects sequences that share at least 50% identity)

Hybridization: 6×SSC at RT to 55° C. for 16-20 hours

Wash at least twice: 2×-3×SSC at RT to 55° C. for 20-30 minutes each.

Increase: To raise the quality, amount, or strength of something. In one example, a therapeutic composition that includes a prn− extract increases the viability of plants or plant cells to one or more stressors, for example as compared to the response in the absence of the prn− extract. In some examples such an increase is evidenced by decreased killing of the plant, seedling, or plant cells. In some examples, the increase in the viability of cells is at least 20%, at least 50%, at least 75%, at least 90%, at least 100%, or even at least 200%, relative to the viability observed when the plant or seedling is untreated or treated with a composition that does not include a prn− extract.

In another example, a prn− plant or seedling has increased production of quercetin, for example as compared to the quercetin produced in the presence of Prn (such as in a wt seedling). In some examples, the increase in quercetin is at least 20%, at least 50%, at least 75%, at least 90%, at least 100%, or even at least 200%, relative to the quercetin produced in a wild-type plant or seedling.

In other examples, increases are expressed as a fold change, such as an increase in the plant or plant cell viability or an amount of quercetin by at least 2-fold, at least 3-fold, at least 4-fold, at least 5-fold, at least 8-fold, at least 10-fold, or even at least 15 or 20-fold, relative to the seedling or plant cell viability or quercetin observed in a seedling that expresses Prn. Such increases can be measured using the methods disclosed herein.

Isolated: An “isolated” biological component (such as a nucleic acid molecule, protein, quercein, or organelle) has been substantially separated or purified away from other biological components in the cell of the organism in which the component naturally occurs, such as other chromosomal and extra-chromosomal DNA and RNA, proteins and organelles. Nucleic acid molecules, proteins, and quercetin that have been “isolated” include nucleic acid molecules and proteins purified by standard purification methods. The term also embraces nucleic acid molecules and proteins prepared by recombinant expression in a host cell as well as chemically synthesized nucleic acid molecules and proteins. In one example, quercetin is isolated from a cell extract, but the resulting quercetin may include other plant components (such as one or more of antibiotic-like derivative compounds, methylsalicyluric acid compound, highly similar to 2S-hydroxy-10-undecanoic acid, piperidine naphthalene-2-carboximida derivative, and a benzoic acid derivative).

Nucleic acid molecule: A deoxyribonucleotide or ribonucleotide polymer in either single or double stranded form, and unless otherwise limited, includes nucleic acid molecules that include analogues of natural nucleotides that can hybridize to nucleic acid molecules in a manner similar to naturally occurring nucleotides. In specific examples, nucleic acid molecules are linear or circular.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as pirin). Generally, operably linked DNA sequences are contiguous and, where necessary to join two protein-coding regions, in the same reading frame.

Promoter: An array of nucleic acid control sequences that directs transcription of a nucleic acid molecule. A promoter includes necessary nucleic acid sequences near the start site of transcription, such as a TATA element. A promoter also optionally includes distal enhancer or repressor elements which can be located as much as several thousand base pairs from the start site of transcription. Both constitutive and inducible promoters are included by this disclosure.

Specific, non-limiting examples of promoters include promoters derived from the genome of a plant cell (such as a ubiquitin promoter or a pirin promoter). Promoters produced by recombinant or synthetic techniques can also be used.

Plant: Refers to either a whole plant, a plant part, a plant cell, or a group of plant cells, such as plant tissue. Plantlets are also included within the meaning of “plant”, as are young plant seedlings. Plants included in the disclosure are any plants amenable to transformation techniques, including angiosperms, gymnosperms, monocotyledons and dicotyledons. Examples of monocotyledonous plants include asparagus, field and sweet corn, barley, wheat, rice, sorghum, onion, pearl millet, rye and oats. Examples of dicotyledonous plants include tomato, tobacco, cotton, potato, rapeseed, field beans, soybeans, peppers, lettuce, peas, alfalfa, clover, cole crops or Brassica oleracea (e.g., cabbage, broccoli, cauliflower, brussels sprouts), radish, carrot, beets, eggplant, spinach, cucumber, squash, melons, cantaloupe, sunflowers and various ornamentals. Woody species include poplar, pine, sequoia, cedar, oak, and the like.

In one example the plant is a highly inbred crop, such as rice, soybean, corn, cotton, wheat, oats, barley, sorghum. In some examples, the plant is one that will likely be exposed to one or more stressors during its growth, such as chilling (cold), heat, salt, high light/UV, flooding, drought/water stress, or predators (e.g., insects, parasitic worms, arachnids).

Particular types of plants include fruit plants (such as strawberry), fruit trees (such as a citrus tree, e.g., orange, lime, lemon or grapefruit tree, as well as other fruit trees, e.g., cherry, papaya or plum tree), flower plants, and grasses. In one example, the plant is a crop plant, such as soybean, corn, canola, tobacco, cotton and the like.

Particular exemplary plants that can be used with the methods provided herein include rice, maize, wheat, barley, sorghum, millet, grass, oats, tomato, corn, potato, banana, kiwi fruit, avocado, melon, mango, cane, sugar beet, tobacco, papaya, peach, strawberry, raspberry, blackberry, blueberry, lettuce, cabbage, cauliflower, onion, broccoli, brussels sprouts, cotton, canola, grape, soybean, oil seed rape, asparagus, beans, carrots, cucumbers, eggplant, melons, okra, parsnips, peanuts, peppers, pineapples, squash, sweet potatoes, rye, cantaloupes, peas, pumpkins, sunflowers, spinach, apples, cherries, cranberries, grapefruit, lemons, limes, nectarines, oranges, pears, tangelos, tangerines, lily, carnation, chrysanthemum, petunia, rose, geranium, violet, gladioli, orchid, lilac, crabapple, sweetgum, maple, poinsettia, locust, ash, linden tree and Arabidopsis thaliana.

In a specific embodiment, a plant cell (e.g., a crop plant such as soybean) includes an isolated nucleic acid molecule that decreases expression or activity of Prn such that the levels of Prn are sufficiently decreased and quercetin is sufficiently increased to protect the plant cell from one or more stressors.

Purified: The term “purified” does not require absolute purity; rather, it is intended as a relative term. Thus, for example, a purified protein or quercetin preparation (such as one generated from a prn− seedling) is one in which the protein or quercetin referred to is more pure than the protein or quercetin in its natural environment within a cell.

Recombinant: A recombinant nucleic acid molecule is one that has a sequence that is not naturally occurring or has a sequence that is made by an artificial combination of two otherwise separated segments of sequence. This artificial combination can accomplished by methods known in the art, such as chemical synthesis or by the artificial manipulation of isolated segments of nucleic acids, e.g., by genetic engineering techniques.

Stress: Refers to one or more stresses that a plant can be exposed to, such as abiotic and biotic stresses. Abiotic stimuli include blue light (which is about 410 nm), ultraviolet (UV) radiation (e.g., UVA which is about 320 nm to 400 nm; UVB which is about 280 nm to 320 nm; and UVC which is about 100 nm to 280 nm), cold, drought, heat, and salt. Biotic stimuli include hormones, fungi, bacteria, arthropods, worms, and products of living organisms. In different tissues of the plant, sufficient levels may be nanomolar quantities, in other tissues they may be micromolar quantities.

Examples of damage resulting from stress include root damage, leaf damage, meristematic damage, shoot damage, inflorescence damage, pod damage, seed damage, and any damage adversely impacting or reducing yield of a plant. In some examples, stress induces increased pigment synthesis by the plant from phenylalanine (Phe). Chronic exposure of a plant to stress can result in reduced photosynthetic capacity, increased susceptibility to disease, reduced biomass yield, reduced seed nutritional content, damage to cellular ultrastructure, and/or an adverse impact on the species interactions and diversity.

Subject or patient: A term that includes human and non-human mammals. In one example, the subject is a human or veterinary subject, such as a mouse. In some examples, the subject is a mammal (such as a human) who has cancer, or is being treated for cancer. in another example, a subject is one in whom it is desired to prevent a fungal infection, such as an immunocompromised subject, for example an HIV infected patient (such as one who has AIDS) or a subject undergoing chemotherapy.

Transformed: A transformed cell is a cell into which a nucleic acid molecule has been introduced, for example by molecular biology techniques. Transformation encompasses all techniques by which a nucleic acid molecule can be introduced into such a cell, including, but not limited to, Agrobacterium-mediated transformation, transfection with viral vectors, transformation with plasmid vectors, and introduction of nucleic acid molecules by electroporation, lipofection, and particle gun acceleration.

Transgene: A nucleic acid sequence that is exogenous to a cell. In one example, a transgene is a vector. In yet another example, the transgene is an RNAi or antisense nucleotide, wherein expression of the antisense or RNAi sequence decreases expression of a target nucleic acid sequence. A transgene can contain regulatory sequences, such as a promoter.

Transgenic cell: Transformed cells which contain foreign, non-native nucleic acid sequences, such as a vector.

Transgenic plant: A plant that contains recombinant genetic material, for example nucleic acid sequences that are not normally found in plants of this type. In a particular example, a transgenic plant includes a vector that has been introduced by molecular biology methods. Includes a plant that is grown from a plant cell into which a recombinant nucleic acid was introduced by transformation, and all offspring of that plant that contain the introduced transgene (whether produced sexually or asexually).

Treating: A term when used to refer to the treatment of a cell or tissue with a therapeutic agent, includes contacting or incubating an agent (such as a prn− or prn+ extract) with the cell or tissue. A treated cell (such as a plant cell or mammalian cell) is a cell that has been contacted with a desired composition in an amount and under conditions sufficient for the desired response. In one example, a treated tumor cell is a tumor cell that has been exposed to a prn− extract until sufficient tumor cell killing is achieved. In one example, a treated plant cell is a plant cell that has been exposed to a prn− extract until sufficient tolerance of the plant cell to a stressor is achieved.

Up-regulated or overexpression: When used in reference to the expression of a nucleic acid molecule, such as a Prn gene, refers to any process which results in an increase in production of a gene product. A gene product can be RNA (such as mRNA, rRNA, tRNA, and structural RNA) or protein. Therefore, gene up-regulation or overexpression includes processes that increase transcription of a gene or translation of mRNA.

Gene up-regulation includes any detectable increase in the production of a gene product, such as Prn protein. In certain examples, production of a gene product increases by at least 20%, at least 50%, or even at least 100%, as compared to a control (such an amount of gene expression in a non-transgenic cell). In one example, a control is a relative amount of gene expression in a corresponding non-transgenic plant of the same variety of the transgenic plant.

Under conditions sufficient for: A phrase that is used to describe any environment that permits the desired activity. In one example, “under conditions sufficient for” includes contacting a plant or plant cell with a prn− extract sufficient to allow the extract to protect the plant or plant cell from stressors.

Untreated cell or plant: A cell or plant that has not been contacted with a desired agent, such as a prn− extract. In an example, an untreated cell is a cell that receives the vehicle (such as a buffer) in which the desired agent was delivered.

Vector: A nucleic acid molecule as introduced into a host cell (such as a plant cell), thereby producing a transformed host cell. A vector may include nucleic acid sequences that permit it to replicate in a host cell, such as an origin of replication. A vector may also include one or more selectable marker genes and other genetic elements known in the art (such as a promoter).

### Overview of the Technology

Phenylpropanoids, such as quercetin, are abundant in young etiolated seedlings, while phenylalanine (Phe), the precursor to many compounds, is present at low levels. The GCR1-GPA1-PD1/ADT3 signaling system is a rapid-response system responsible for the enhanced production of Phe and phenylpropanoids like quercetin, which absorbs UV-B and exhibits strong anti-oxidant capabilities, where it may reduce/prevent damaging effects of abiotic and biotic stress. Pirin1 (PRN1) expressed in etiolated Arabidopsis functions as a GCR1-GPA1 effector, regulating ABA and BL-mediated gene expression via interaction with NFY and the CCAAT box located in several ABA- and BL-responsive genes (Warpeha et al., Plant Physiol. 140: 844-55, 2006; Warpeha et al., Plant Physiol. 144:1590-1600, 2007). It is shown herein that in vitro-synthesized Arabidopsis PRN1 has quercetinase activity regulated through its interaction with GPA1. The quercetinase activity of PRN1 is turned off as a result of interaction with activated GPA1. Elimination of the PRN1 gene leads to high levels of quercetin in etiolated seedlings, while levels of closely related compounds are unchanged.

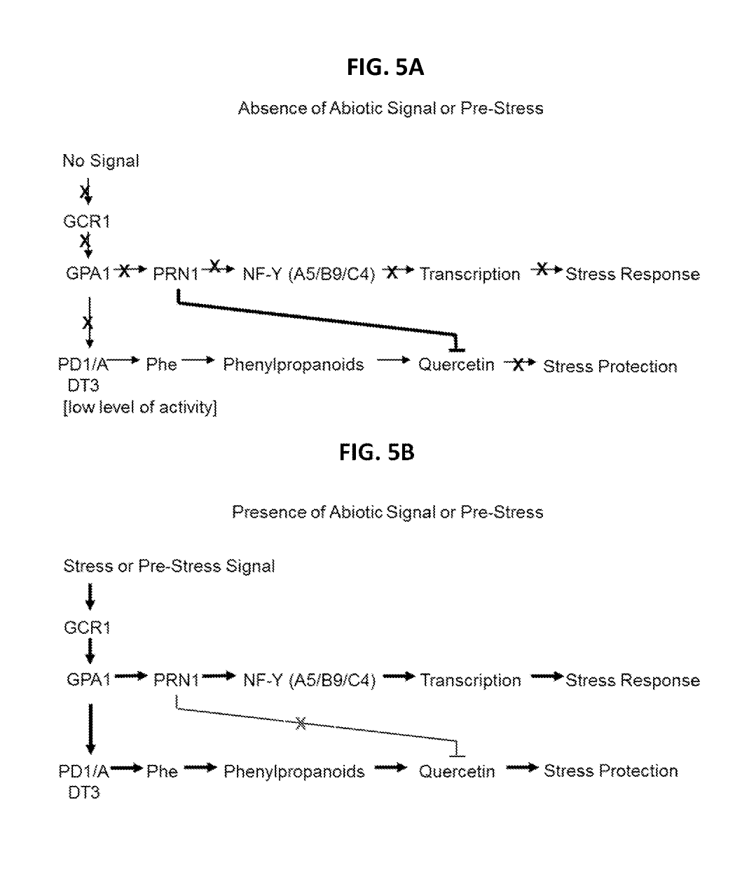

Based on the data provided herein, the following model is proposed. As shown in FIG. 5A, in the absence of stimulation by an abiotic signal, GPA1 interacts with Prn which functions as a quercetinase and breaks down the phenylpropanoid quercetin, which can leave the seedling susceptible to stress. Thus, in etiolated Arabidopsis seedlings, PD1/ADT3 is inactive, producing only low levels of Phe and quercetin. PRN1 acts as a quercetinase actively degrading any quercetin that might be made. As shown in FIG. 5B, in the presence of an abiotic signal (e.g., salt, heat, UV-B), activates the GCR1-GPA1 pathway through one or several phytohormone second messengers (e.g., abscisic acid (ABA), ethylene (ET), jasmonic acid (JA), salicylic acid (SA)). These second messengers, alone or in combination, lead to the activation of GCR1. GCR1 activates GPA1, which in turn activates PD1/ADT3, leading to the activation of PD1/ADT3, enhanced Phe synthesis, and thereby enhanced quercetin synthesis. Simultaneously, Prn quercetinase activity is switched off, allowing Prn to interact with NFY to carry out its role as a transcriptional activator.

### Pirin Sequences

Pirin (Prn or Pir) is an iron binding protein of the cupin superfamily characterized by small beta-barrel folds. It is found in humans, plants, and prokaryotes. Pirin binds to NF-Y (CBF; Hap2/3/5) transcription factors (CCAAT). Pirin activity includes the ability of a pirin protein to function as a quercetinase, that is, the ability to degrade the phenylpropanoid quercetin. In one example, pirin activity it is the ability of pirin to increase the tolerance of a plant to abiotic (and in some examples biotic) stressors. In one example, such activity occurs in a cell, such as a plant cell. In another example, such activity occurs in vitro. Such activity can be measured using any assay known in the art, for example the assays described below in EXAMPLE 1.

Prn nucleic acid coding sequences and protein sequences are publicly available, for example from GenBank. However, the disclosure is not limited to the use of particular Prn sequences. In one example, the Prn sequence used is a plant Prn sequence (such as a Prn1 sequence). Exemplary Prn sequences include those available from GenBank, for example, GenBank Accession Nos. CP002686 and NM_{—}115784.2 disclose Arabadopsis thaliana Prn1 nucleic acid sequences; GenBank Accession Nos. AEE79893.1 and NP_{—}191481 disclose Arabadopsis thaliana Prn1 protein sequences; GenBank Accession Nos. NM_{—}003662, Y07867 and NM_{—}001018109.2 disclose human Prn nucleic acid sequences; GenBank Accession Nos. NP_{—}003653, CAA69194 and NP_{—}001018119 disclose human Prn protein sequences; GenBank Accession Nos. DP000009 and ABF99862 disclose Oryza sativa Prn nucleic acid and protein sequences, GenBank Accession Nos. FN596745.1 and CBI39450 disclose Vitis vinifera Prn nucleic acid and protein sequences, and GenBank Accession Nos. BT098660 and ACU23860 disclose soybean Prn nucleic acid and protein sequences, respectively. Other specific pirin sequences are provided in SEQ ID NOS: 1-16.

In particular examples, a pirin nucleic acid sequence includes the sequence shown in SEQ ID NO: 1, 3, 11, 13, or 15, or variants thereof that retain the ability to encode a protein having pirin activity (such as a sequence having at least 80%, at least 85%, at least 90%, at least 95%, at least 97%, at least 98%, or at least 100% sequence identity to SEQ ID NO: 1, 3, 11, 13, or 15 or any of the nucleic acid GenBank numbers provided herein). In another example, a pirin protein includes the amino acid sequence shown in any of SEQ ID NOS: 2, 4-10, 12, 14, or 16, or variants thereof that retain pirin activity (such as a sequence having at least 80%, at least 85%, at least 90%, at least 95%, at least 97%, at least 98%, or at least 100% sequence identity to SEQ ID NO: 2, 4, 5, 6, 7, 8, 9, 10, 12, 14, or 16).

“Sequence identity” is a phrase commonly used to describe the similarity between two or more nucleic acid or amino acid sequences. Sequence identity typically is expressed in terms of percentage identity; the higher the percentage, the more similar the sequences. Methods for aligning sequences for comparison and determining sequence identity are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. USA, 85:2444, 1988; Higgins and Sharp, Gene, 73:237-244, 1988; Higgins and Sharp, CABIOS, 5:151-153, 1989; Corpet et al., Nucleic Acids Research, 16:10881-10890, 1988; Huang, et al., Computer Applications in the Biosciences, 8:155-165, 1992; Pearson et al., Methods in Molecular Biology, 24:307-331, 1994; Tatiana et al., FEMS Microbiol. Lett., 174:247-250, 1999. Altschul et al. present a detailed consideration of sequence-alignment methods and homology calculations (J. Mol. Biol., 215:403-410, 1990).

The National Center for Biotechnology Information (NCBI) Basic Local Alignment Search Tool (BLAST™, Altschul et al., J. Mol. Biol., 215:403-410, 1990) is publicly available from several sources, including the National Center for Biotechnology Information (NCBI, Bethesda, Md.) and on the Internet, for use in connection with the sequence-analysis programs blastp, blastn, blastx, tblastn and tblastx. A description of how to determine sequence identity using this program is available on the internet under the help section for BLAST™.

BLASTN is used to compare nucleic acid sequences, while BLASTP is used to compare amino acid sequences. To compare two nucleic acid sequences, the options can be set as follows: -i is set to a file containing the first nucleic acid sequence to be compared (such as C:\seq1.txt); -j is set to a file containing the second nucleic acid sequence to be compared (such as C:\seq2.txt); -p is set to blastn; -o is set to any desired file name (such as C:\output.txt); -q is set to −1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q −1-r 2.

To compare two amino acid sequences, the options of Bl2seq can be set as follows: -i is set to a file containing the first amino acid sequence to be compared (such as C:\seq1.txt); -j is set to a file containing the second amino acid sequence to be compared (such as C:\seq2.txt); -p is set to blastp; -o is set to any desired file name (such as C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least 60%, at least at least 70%, at least 80%, at least 90%, at least 95%, at least 98%, or at least 99% sequence identity determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

In addition to the specific sequences provided herein, and the sequences which are currently publicly available, one skilled in the art will appreciate that variants of such sequences can be used. For example, a Prn sequence may vary between different organisms. In particular examples, a variant Prn sequence retains the biological activity of its corresponding native Prn sequence.

One of ordinary skill in the art will appreciate that a DNA sequence can be altered in numerous ways without affecting the biological activity of DNA sequences. For example, a variant sequence can optimized for expression in a particular cell (e.g., by optimizing codon usage). In one example, a variant is a sequence change to a DNA sequence. Two types of DNA sequence variant can be produced. In the first type, the variation in the DNA sequence is not manifested as a change in the amino acid sequence of the encoded peptide. These silent variations reflect the degeneracy of the genetic code. In the second type, the DNA sequence variation changes the amino acid sequence of the encoded protein. In such cases, the variant DNA sequence produces a variant peptide sequence. In order to optimize preservation of the functional and immunologic identity of the encoded polypeptide, any such amino acid substitutions can be conservative. Conservative substitutions replace one amino acid with another amino acid that is similar in size, hydrophobicity, and so forth. Such substitutions generally are conservative when it is desired to finely modulate the characteristics of the protein.

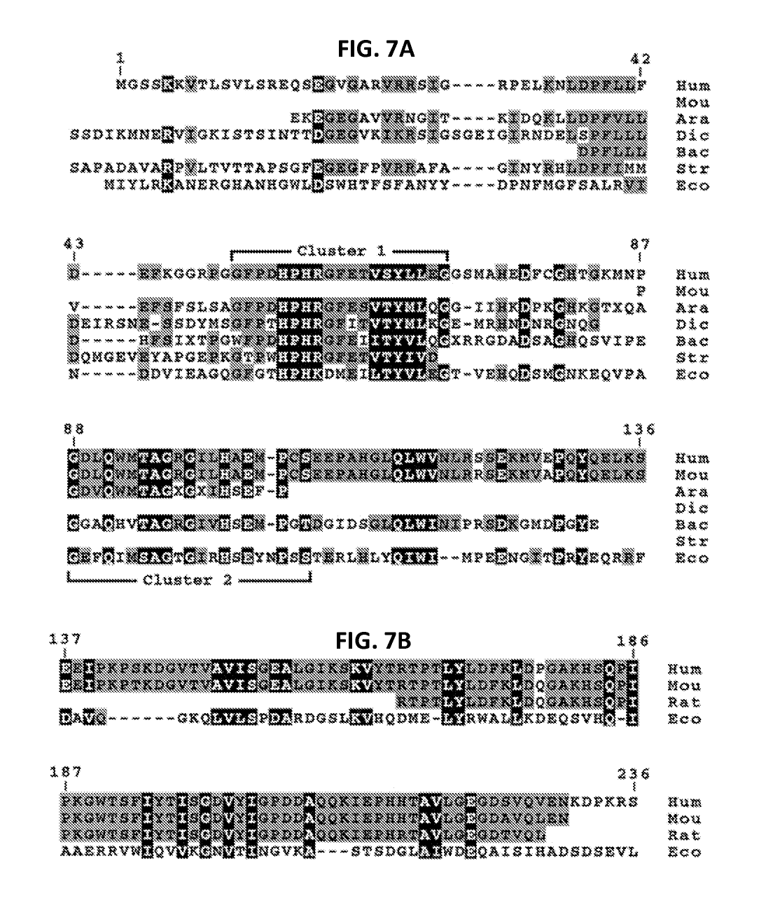

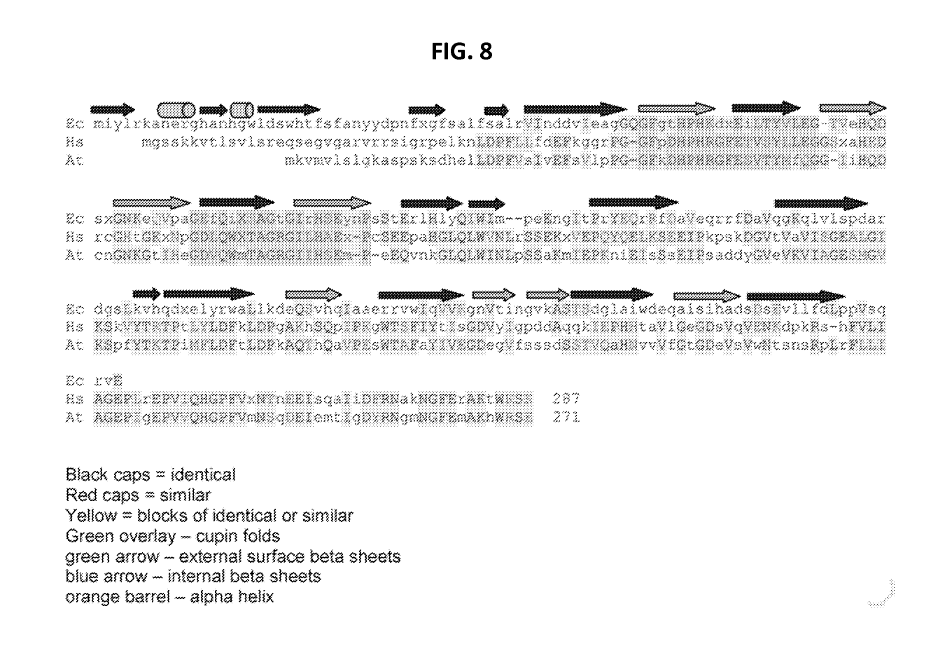

In some examples, variations in the Prn DNA sequence that result in amino acid changes, whether conservative or not, are minimized to enhance preservation of the functional and immunologic identity of the encoded Prn protein. In particular examples, a DNA sequence variant will introduce no more than 20, for example fewer than 10 amino acid substitutions into the encoded Prn polypeptide, such as 1-5 or 1-10 amino acid substitutions. Variant amino acid sequences can, for example, be at least 80%, at least 90% or even at least 95% identical to the native amino acid sequence. For example, a Prn sequence can be used that has conservative amino acid changes (such as, very highly conserved substitutions, highly conserved substitutions or conserved substitutions), such as 1 to 5, 1 to 20, or 1 to 10 conservative amino acid substitutions, such as 1, 2, 3, 4, 5, 6, 7, 8, 9, 10, 15 or 20 conservative amino acid substitutions (for example this number of conservative amino acid substitutions in any of SEQ ID NOS: 2, 4, 5, 6, 7, 8, 9, 10, 12, 14, or 16). Conserved residues in the same or similar proteins from different species can also provide guidance about possible locations for making substitutions in the sequence. For example, a Prn residue which is highly conserved across several species is more likely to be important to the function of the Prn protein than a residue that is less conserved across several species (see FIGS. 7A, 7B and 8 for example).

Exemplary conservative amino acid substitutions that can be made to any of SEQ ID NOS: 2, 4, 5, 6, 7, 8, 9, 10, 12, 14, or 16 are shown in Table 1.

TABLE 1
Exemplary conservative amino acid substitutions.

| | | Highly Conserved | Conserved |
|---|---|---|---|
| | Very Highly - | Substitutions | Substitutions |
| Original | Conserved | (from the | (from the |
| Residue | Substitutions | Blosum90 Matrix) | Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, | Arg, Asp, Gln, Glu, |
| | | Lys, Ser, Thr | His, Lys, Ser, Thr |
| Asp | Glu | Asn, Glu | Asn, Gln, Glu, Ser |
| Cys | Ser | None | Ala |
| Gln | Asn | Arg, Asn, Glu, | Arg, Asn, Asp, Glu, |
| | | His, Lys, Met | His, Lys, Met, Ser |
| Glu | Asp | Asp, Gln, Lys | Arg, Asn, Asp, Gln, |
| | | | His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, |
| | | | Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

### Extracts from prn− Mutant Plants and Methods of Use

Plants or seedlings that are functionally deleted (genetically inactivated) for Pirin or Pirin1, referred to herein as prn− or prn1− plants, have significantly increased levels of quercetin. In addition, such plants or seedlings have increased tolerance to stressors, such as UV light. As a result, a plant or seedling can be made prn− to enhance its tolerance to one or more stressors. In addition, such prn− plants or seedlings can be used for making extracts that contain quercetin. The resulting extracts can be used for a variety of purposes, such as making quercetin, protecting plants from biotic or abiotic stressors (or increasing their tolerance from such stressors), treating tumor cells, preventing infection in a subject by a toxic fungus such as C. gattii, preventing a toxic fungus such as C. gattii from growing on a surface, and increasing anti-oxidant activity in a subject.

A. Transgenic prn− Plants

Transgenic plants, seedlings, and plant cells that are prn− can be generated using routine methods in the art. For example, such plants, seedlings, and cells can include one or more exogenous nucleic acid molecules that decrease pirin activity in the cell, thereby decreasing or even eliminating functional pirin protein in the cell. However, 100% inactivation is not required, as long as levels of quercetin in the plant increase and the levels of pirin expressed are decreased sufficiently to protect the plant cell from damage from an abiotic or biotic stressor, such as UV light. For example, prn− plants, seedlings, and cells can have at least a 50% decrease in detectable Prn, such as at least at 75%, at least 80%, at least 85%, at least 90%, at least 95% or even an at least 99% decrease in Prn levels. In some examples, Prn expression or activity is completely eliminated.

Because pririn is a quercetinase, the result of decreasing or inactivating pirin in the plant cell is a transgenic plant, seedling, or cell having increased levels of quercetin as compared to a comparable non-transgenic plant, seedling, or cell, such as at least 2 times, at least 2.5 times, at least 3 times, at least 3.5 times, or at least 4 times more quercetin, such as 2 to 10, 2 to 5, or 2 to 4 times more quercetin.

Methods of functionally deleting or inactivating a gene in a plant, seedling, and cell are routine, and the disclosure is not limited to particular methods of making prn− plants, seedlings, and cells. However, exemplary methods are provided below.

1. RNAi

Inhibitory RNA (RNAi) constructs can be used to decrease or inhibit expression of any plant Prn sequence, such as decrease or inhibit expression of the protein shown in SEQ ID NO: 2, 4, 5, 6, 7, 8, 9, 10, 12, 14, or 16 (or a sequence having at least 80%, at least 85%, at least 90%, at least 95%, at least 97%, or at least 98% sequence identity to SEQ ID NO: 2, 4, 5, 6, 7, 8, 9, 10, 12, 14, or 16). One skilled in the art will understand that RNAi constructs can be generated to any plant Prn sequence. In particular examples, an RNAi construct includes a DNA sequence that is a portion of a plant Prn sequence, arranged in sense and antisense orientations under the control of a promoter. The transcription of the sense and the antisense DNA sequence results in a dsRNA, then siRNA. The siRNA molecule can cause sequence-specific destruction of mRNAs, allowing targeted knockdown of gene expression. In one example, a DNA sequence used for an RNAi construct is specific for SEQ ID NO: 1, 3, 11, 13, or 15 (or a sequence having at least 80%, at least 85%, at least 90%, at least 95%, at least 97%, or at least 98% sequence identity to SEQ ID NO: 1, 3, 11, 13, or 15). This disclosure is not limited to RNAi compounds of a particular length. A DNA sequence used for an RNAi construct can be any length, such as at least 100 base pairs (bp), at least 200 bp, at least 300 bp, or even at least 400 bp, such as 100 to 1000 bp or 100 to 500 bp.

For example, a 200 bp DNA sequence can be used to generate an RNAi construct. In particular examples, this RNAi construct is introduced into a plant cell, such as a cell of a plant in which an extract is to be generated, or a plant in which increased tolerance to one or more stressors is desired. Such methods will result in production of an siRNA molecule that will decrease Prn expression

2. Antisense Nucleic Acid Molecules

One approach to disrupting Prn expression is to use antisense oligonucleotides. To design antisense oligonucleotides, a Prn mRNA sequence, such as a plant Prn sequence, is examined. Regions of the sequence containing multiple repeats, such as TTTTTTTT, are not as desirable because they will lack specificity. Several different regions can be chosen. Of those, oligos are selected by the following characteristics: those having the best conformation in solution; those optimized for hybridization characteristics; and those having less potential to form secondary structures. Antisense molecules having a propensity to generate secondary structures are less desirable.

Plasmids or vectors including the antisense sequences of a Prn sequence can be generated. For example, cDNA fragments or variants coding for a Prn protein (such as a sequence having at least 80% sequence identity to SEQ ID NO: 2, 4, 5, 6, 7, 8, 9, 10, 12, 14 or 16 such as at least 85%, at least 90%, at least 95%, at least 97%, or at least 98% sequence identity to SEQ ID NO: 2, 4, 5, 6, 7, 8, 9, 10, 12, 14 or 16) can be PCR amplified and cloned in antisense orientation in a vector. The nucleotide sequence and orientation of the insert can be confirmed by sequencing using a Sequenase kit (Amersham Pharmacia Biotech).

Generally, the term “antisense” refers to a nucleic acid molecule capable of hybridizing to a portion of a Prn RNA (such as mRNA) by virtue of some sequence complementarity. The antisense nucleic acid molecules disclosed herein can be oligonucleotides that are double-stranded or single-stranded, RNA or DNA or a modification or derivative thereof, which can be incorporated into a vector and transfected into a plant or plant cell, to permit expression of the antisense sequence in the cell.

Prn antisense nucleic acid molecules are polynucleotides, and can include sequences that are at least 6 bp in length. In particular examples, antisense sequences range from about 6 to about 500 bp in length, such as 6 to 100 bp or 6 to 50 bp. A Prn antisense polynucleotide recognizes any species of a plant Prn gene sequence. In specific examples, the polynucleotide is at least 10, at least 15, at least 100, at least 200, or at least 500 bp. However, antisense nucleic acid molecules can be much longer. The nucleotides of the antisense sequence can be modified at the base moiety, sugar moiety, or phosphate backbone, and can include other appending groups such as peptides, or agents facilitating transport across the cell membrane.

A Prn antisense polynucleotide, such as a single-stranded DNA, can be modified at any position on its structure with substituents generally known in the art. For example, a modified base moiety can be 5-fluorouracil, 5-bromouracil, 5-chlorouracil, 5-iodouracil, hypoxanthine, xanthine, acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl)uracil, and 2,6-diaminopurine.

In another example, a Prn antisense molecule includes at least one modified sugar moiety such as arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In yet another example, a Prn antisense molecule is an α-anomeric oligonucleotide. An α-anomeric oligonucleotide forms specific double-stranded hybrids with complementary RNA in which, contrary to the usual J-units, the strands run parallel to each other (Gautier et al., Nucl. Acids Res. 15:6625-41, 1987). The oligonucleotide can be conjugated to another molecule (such as a peptide, hybridization triggered cross-linking agent, transport agent, or hybridization-triggered cleavage agent). Oligonucleotides can include a targeting moiety that enhances uptake of the molecule by cells. The targeting moiety can be a specific binding molecule, such as an antibody or fragment thereof that recognizes a molecule present on the surface of the cell, such as a plant cell.

Antisense molecules can be synthesized by standard methods, for example by use of an automated DNA synthesizer. As examples, phosphorothioate oligos can be synthesized by the method of Stein et al. (Nucl. Acids Res. 1998, 16:3209), methylphosphonate oligos can be prepared by use of controlled pore glass polymer supports (Sarin et al., Proc. Natl. Acad. Sci. USA 85:7448-51, 1988). In a specific example, an antisense oligonucleotide that recognizes a Prn sequence includes catalytic RNA, or a ribozyme (see WO 90/11364, Sarver et al., Science 247:1222-5, 1990). In another example, the oligonucleotide is a 2′-O-methylribonucleotide (Inoue et al., Nucl. Acids Res. 15:6131-48, 1987), or a chimeric RNA-DNA analogue (Inoue et al., FEBS Lett. 215:327-30, 1987).

The antisense nucleic acids disclosed herein include a sequence complementary to at least a portion of an RNA transcript of a Prn gene. However, absolute complementarity, although advantageous, is not required. A sequence can be complementary to at least a portion of an RNA; in the case of double-stranded antisense nucleic acids, a single strand of the duplex DNA can thus be tested, or triplex formation can be assayed. The ability to hybridize depends on the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The relative ability of polynucleotides to bind to complementary strands is compared by determining the Tₘ of a hybridization complex of the poly/oligonucleotide and its complementary strand. The higher the Tₘ the greater the strength of the binding of the hybridized strands. As close to optimal fidelity of base pairing as possible achieves optimal hybridization of an oligonucleotide to its target RNA.

3. Site-Specific DNA Recombination

Site-specific DNA recombination can be used to produce transgenic plants that have reduced Prn activity, and thus increased quercetin production. Site-specific recombination is a process involving reciprocal exchange between specific DNA recombining sites catalyzed by recombinases. Site-specific recombinases recognize specific DNA sequences, and in the presence of two such recombination sites, catalyze the recombination of DNA strands. Recombinases can catalyze excision or inversion of a DNA fragment according to the orientation of their specific target sites. Recombination between directly oriented sites leads to excision of the DNA between them, whereas recombination between inverted target sites causes inversion of the DNA between them. Some site-specific recombination systems do not require additional factors for their function and are capable of functioning accurately and efficiently in various heterologous organisms.

One particular example of a site-specific recombination system is the Cre/lox system of bacteriophage P1. Cre recombinase can excise, invert, or integrate extrachromosomal DNA molecules in plant cells. Another particular example of a site-specific recombination system is the FLP/FRT recombination system of yeast. The recombinase FLP can catalyze efficient recombination reactions in heterologous eukaryotic cells. The in planta functionality of FLP/FRT system has been previously demonstrated in Arabidopsis for excisional recombination and in rice. Therefore, a recombination system, such as the FLP/FRT recombination system, can be used to control, through hybridization to FLP-expressing plants, the down-regulation of a plant Prn gene, producing transgenic plants with increased quercetin.

A particular example of using site-specific DNA recombination to reduce transgene escape includes the following. The first plant includes a first vector, wherein the first vector includes a promoter operably linked to a blocking sequence, and the blocking sequence is flanked by a recombining site sequence. The first vector also includes one or more nucleic acid sequences that reduce expression of a Prn gene. Such nucleic acid sequences are downstream of the blocking sequence such that the nucleic acid sequence that reduces expression of a Prn gene is operably linked to the promoter upon recombination of the recombining site sequence

The second plant includes a second vector which includes a recombinase, such as a promoter operably linked to a recombinase. In particular examples, the recombinase is integrated in the genome of the second plant. The method includes permitting expression of the recombinase in the second plant, or permitting expression of the recombinase in the resulting hybrid progeny of the first and second plants. Expression of the recombinase will remove the blocking sequence from the first vector, resulting in the promoter being operably linked to the nucleic acid sequence that reduces expression of a Prn gene. Expression of the nucleic acid sequence that reduces expression of a Prn gene results in production of a transgenic plant with increased quercetin and increased tolerance to stressors. The second vector can further include a promoter operably linked to a selectable marker.

The promoter operably linked to the recombinase can be a constitutive promoter, such as a ubiquitin promoter, for example a rice ubiquitin promoter. In other examples, the promoter operably linked to the recombinase is an inducible promoter, and permitting expression of the recombinase includes contacting the second plant with an inducing agent (thereby activating the inducible promoter). Exemplary inducible promoters include, but are not limited to, a heat shock promoter, a chemically inducible promoter, or a light activated promoter. The inducing agent (such as heat, a chemical, or light) can be contacted with the second plant before or during crossing with the first fertile plant, or can be contacted with the resulting hybrid progeny following the crossing.

Exemplary recombinases and recombining sites include, but are not limited to: FLP/FRT, CRE/lox, R/RS sequence, and Gin/gix. Blocking sequences are known in the art, and include selectable marker gene sequences, such as a hyg, or bar, or pat cDNA sequence.

B. Generation of Extracts from prn− Plants

The transgenic prn− plants described above can be used for making extracts. Thus, extracts generated by such plants or seedlings are contemplated by this disclosure. In some examples, the method of making an extract includes extracting aerial portions from a transgenic seedling (such as a cotyledon). For example, the extracts can be generated from aerial portions (cotyledons or cotyledons+stem) of seedlings that were grown in the dark, exposed to a stressor, and are 7 days or less old.

In some examples, seeds of prn− mutants (such as null prn1 mutants) are planted then grown in complete darkness. In some examples, the seeds are sterilized and rinsed in complete darkness before they are sown. In some examples, the seeds are planting in the morning (such as between 8-11 am or 9-10 am). Seeds can be maintained in the cold, for example at −0° C. to 5° C., for example 2° C. to 4° C., such as 4° C., for at least 24 hours, at least 36 hours, or at least 48 hours, such as 24 to 72 hours or 24 to 48 hours, such as 48 hours, in a sealed dark container (no light penetration). The seeds are then moved to complete darkness at about room temperature, for example at least 15° C., or at least 20° C., such as 15° C. to 25° C., or 15° C. to 20° C., such as 20° C., for a period of at least 4 days, such as at least 5 days, or at least 6 days, such as 4 to 8 days, 5 to 7 days, or 5 to 6 days, such as 5, 6, or 7 days. In a specific example, seeds are maintained for 48 h in 4° C. in a sealed dark container (no light penetration), then moved to 20° C. for 6 days in complete darkness.

Following growth in darkness, the transgenic seedlings are exposed to one or more abiotic or biotic stressors. For example, the transgenic plant can be exposed to UV radiation (e.g., UV-A, B, or C), cold, drought, heat, salt, hormones, or combinations thereof. Table 2 provides exemplary stressor conditions.

TABLE 2
Exemplary Stressor conditions

| Stressor | Conditions |
|---|---|
| UV A radiation | 10⁴ μmolm⁻² in less than 5 min not more than 10 min |
| UV B radiation | 10⁴ μmolm⁻² in less than 10 min not less than 20 min |
| UV C radiation | 10⁴ μmolm⁻² in less than 10 min |
| Cold | 2-5° C. given in 40-60 min |
| Heat | 48-52° C. given in 40-60 min |
| Drought | −15-30% water, elevated temperature 40-50° C. given in |
| | 40-60 min, 100 mM NaCl |
| Salt | 150 mM NaCl given in 2-6 hours |
| Hormones | 1 μM |

After exposing the transgenic plant to one or more stressors, the plant is immediately returned to complete darkness. In a specific example, on day 6, seedlings are given a total dose of 10⁴ μmolm⁻² of 317 nm (UVB) for 10 minutes with no other irradiation, or 10⁴ μmolm⁻² 254 nm UVC for 4 minutes, and immediately returned to complete darkness. Subsequently, aerial portions of seedlings including the cotyledons are harvested, for example under a dim green light of 0.1 μmolm⁻². In particular examples, the aerial portions of seedlings are harvested at least 6 hours later, such as at least 12 hours later, at least 18 hours later, at least 24 hours later, such as 12 to 24 hours later, 12 to 28 hours later, 18 to 24 hours later, or 20-24 hours later, such as 24 hours later. In a specific example, the aerial portions of seedlings are harvested 24 hours after treatment with the stressor under a dim green light of 0.1 μmolm⁻².

The aerial portions of seedlings can be harvested into a buffer, such as a buffer containing 20 mM K₂PO₄ pH 7.5 or HEPES pH 7.5, 10 mM NaCl, 1.0 mM dithiothreitol, and a 0.1% protease inhibitor cocktail for plants, at a ratio of 1 part plant material to 9 parts buffer by volume. The aerial portions are then ground up or homogenized until all material is smashed (for example for at least 1 minute, at least 3 minutes, at least 5 minutes, or at least 10 minutes, such as 1 to 5 minutes). In one example the aerial portions of seedlings are homogenized in the buffer in a bullet point glass tissue homogenizer for 5 minutes.

The resulting homogenate can then be processed to remove solid materials. For example, the homogenate can be spun at 4° C. in darkness for 5,000 rpm in a microfuge in non-extractable plastic tubes. The resulting supernatant is removed and stored in darkness at 4° C. Prior to use, the extract can be warmed, for example to room temperature, such as at least 15° C., or at least 20° C., such as 15° C. to 25° C., or 15° C. to 20° C., such as 20° C. In some examples, the extract is diluted prior to use, such as 1/20 or 1/100 volume. It was observed that the extract stayed effective when stored in the dark at 4° C., and used within 14 days.

1. Extract-Containing Compositions

The resulting transgenic plant extract can be used directly, can be concentrated, diluted in one or more pharmaceutically acceptable carriers, or combinations thereof. The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of therapeutic compounds, such as the extracts provided herein. In particular examples, the extract is present in water or physiological saline.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some examples, the extract further includes one or more chemotherapeutic agents. Chemotherapeutic agents include agents with therapeutic usefulness in the treatment of diseases characterized by abnormal cell growth. For example, chemotherapeutic agents are useful for the treatment of cancer, including breast cancer. In one embodiment, a chemotherapeutic agent is a radioactive compound. Another example includes tyrosine kinase inhibitors, such as lapatinib. In particular examples, such chemotherapeutic agents decrease or reduces homo- or heterodimerization of HER proteins (for example before antibodies that specifically bind to HER2 or conjugate thereof, such as Herceptin®). One of skill in the art can readily identify an appropriate chemotherapeutic agent to use in combination with the prn− extract, depending on the tumor to be treated (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., ©2000 Churchill Livingstone, Inc; Baltzer, L., Berkery, R. (eds): Oncology Pocket Guide to Chemotherapy, 2nd ed. St. Louis, Mosby-Year Book, 1995; Fischer, D.S., Knobf, M. F., Durivage, H.J. (eds): The Cancer Chemotherapy Handbook, 4th ed. St. Louis, Mosby-Year Book, 1993; Chabner and Longo, Cancer Chemotherapy and Biotherapy: Principles and Practice (4th ed.). Philadelphia: Lippincott Willians & Wilkins, 2005; Skeel. Handbook of Cancer Chemotherapy (6th ed.). Lippincott Williams & Wilkins, 2003).

Other particular examples of therapeutic agents that can be combined with a prn− extract include microtubule binding agents, DNA intercalators or cross-linkers, DNA synthesis inhibitors, DNA and/or RNA transcription inhibitors, antibodies, enzymes, enzyme inhibitors, gene regulators, and angiogenesis inhibitors. These agents (which are administered at a therapeutically effective amount) and treatments can be used alone or in combination. Methods and therapeutic dosages of such agents are known to the person of ordinary skill in the art, and can be determined by a the person of ordinary skill in the art.

Microtubule binding agent refers to an agent that interacts with tubulin to stabilize or destabilize microtubule formation thereby inhibiting cell division. Examples of microtubule binding agents that can be used in conjunction with the disclosed therapy include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and are known to those of ordinary skill in the art. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

Suitable DNA and/or RNA transcription regulators, including, without limitation, actinomycin D, daunorubicin, doxorubicin and derivatives and analogs thereof also are suitable for use in combination with the disclosed therapies. DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, formestane, trichostatin and derivatives and analogs thereof. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof.

Examples of the commonly used chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

Non-limiting examples of anti-angiogenic agents include molecules, such as proteins, enzymes, polysaccharides, oligonucleotides, DNA, RNA, and recombinant vectors, and small molecules that function to reduce or even inhibit blood vessel growth. Examples of suitable angiogenesis inhibitors include, without limitation, angiostatin K1-3, staurosporine, genistein, fumagillin, medroxyprogesterone, suramin, interferon-alpha, metalloproteinase inhibitors, platelet factor 4, somatostatin, thromobospondin, endostatin, thalidomide, and derivatives and analogs thereof. For example, in some embodiments the anti-angiogenesis agent is an antibody that specifically binds to VEGF (e.g., Avastin, Roche) or a VEGF receptor (e.g., a VEGFR2 antibody). In one example the anti-angiogenic agent includes a VEGFR2 antibody, or DMXAA (also known as Vadimezan or ASA404; available commercially, e.g., from Sigma Corp., St. Louis, Mo.) or both. Exemplary kinase inhibitors include Gleevac, Iressa, and Tarceva that prevent phosphorylation and activation of growth factors. Antibodies that can be used include Herceptin and Avastin that block growth factors and the angiogenic pathway.

In some examples, the therapeutic agent is a monoclonal antibody, for example, 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, sorafenib, sunitinib, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab.

Other therapeutic agents, for example anti-tumor agents, that may or may not fall under one or more of the classifications above, also are suitable for administration in combination with the disclosed therapies. By way of example, such agents include adriamycin, apigenin, rapamycin, zebularine, cimetidine, and derivatives and analogs thereof.

In some examples, the prn− extract further includes one or more anti-fungal agents, such as a polyene antifungal (for example Natamycin, Rimocidin, Filipin, Nystatin, Amphotericin B, Candicin or Hamycin), an imidazole (for example Miconazole, Ketoconazole, Clotrimazole, Econazole, Omoconazole, Bifonazole, Butoconazole, Fenticonazole, Isoconazole, Oxiconazole, Sertaconazole, Sulconazole, Tioconazole), a thiazole (for example Fluconazole, Itraconazole, Isavuconazole, Ravuconazole, Posaconazole, Voriconazole, Terconazole), a thiazole, an allylamine (Terbinafine, Naftifine, or Butenafine), or an echinocandin. In some examples, the prn− extract further includes agents that are used to treat C. gatti infections, such as amphotericin B and flucytosine.

In some examples, the prn− extract further includes one or more antioxidants, such as on or more of glutathione, beta carotene, vitamin C, vitamin E, enzymes (such as catalase, superoxide dismutase and peroxidases), lipoic acid, carotenes, coenzyme Q, uric acid, melatonin, polyphenol, and reservatrol.

In some examples, the prn− extract further includes one or more agents appropriate for a sunscreen, such as at least one UVA filter and/or at least one UVB filter and/or at least one inorganic pigment, such as an inorganic micropigment. The UVB filters can be oil-soluble or water-soluble. Oil-soluble UVB filter substances can include, for example: 3-benzylidenecamphor derivatives, such as 3-(4-methylbenzylidene)camphor and 3-benzylidenecamphor; 4-aminobenzoic acid derivatives, such as 2-ethylhexyl 4-(dimethylamino)benzoate and amyl 4-(dimethylamino)benzoate; esters of cinnamic acid, such as 2-ethylhexyl 4-methoxycinnamate and isopentyl 4-methoxycinnamate; derivatives of benzophenone, such as 2-hydroxy-4-methoxybenzophenone, 2-hydroxy-4-methoxy-4′-methylbenzophenone and 2,2′-dihydroxy-4-methoxybenzophenone; esters of benzalmalonic acid, such as di(2-ethylhexyl)-4-methoxybenzalmalonate. Water-soluble UVB filter substances can include the following: salts of 2-phenylbenzimidazole-5-sulphonic acid, such as its sodium, potassium or its triethanolammonium salt, and the sulphonic acid itself; sulphonic acid derivatives of benzophenones, such as 2-hydroxy-4-methoxybenzophenone-5-sulphonic acid and salts thereof; sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)benzenesulphonic acid and salts thereof.

2. Surfaces Coated with prn− Extract

The prn− extracts generated from prn− plants provided herein can be used to coat a surface. For example, coating surfaces can be used to prevent toxic fungal infections, such as infections with C. gattii. In some examples, coating surfaces can be used to reduce or inhibit growth of toxic fungi, (such as a reduction in growth of at least 20%, at least 50%, at least 75% or at least 90%) such as C. gattii. In some examples prn− extracts can be applied to surfaces that may come in contact with a toxic fungus, such as C. gattii. For example, prn− extracts can be applied to surfaces in homes and hospitals (or other medical facilities, such as nursing homes and clinics), as well as soil. In some examples, prn− extracts can be applied to walls, floors, counter surfaces, bed rails, medical equipment, and the like. In some examples, prn− extracts can be added to you bags of soil to protect plants from fungal infection or increase their tolerance to stress. Ground soils, such as those in the Pacific Northwest, can be treated with a prn− extracts to reduce the growth of C. gattii. In some examples, prn− extracts can be applied to the inside of a planting container to protect plants from fungal infection or increase their tolerance to stress.

In some examples, the prn− extract is applied as a protective coating to a plastic surface, such as a polymer surface, to protect the plastic from UV damage, such as a plastic exposed to the outdoors for a long period of time.

The prn− extracts generated from prn− plants provided herein can also be used to coat a plastic material, such as one that will be inserted to delivered to a patient. Thus, provided herein are compositions that include a plastic material, which contains a prn− extract on its surface. In some examples, the plastic material is coated with the extract to prevent infection by a toxic fungus such as C. gattii. In one example the material is a polymer, such as one that includes polyolefin, styrene, vinyl, polyamide, polyester, polycarbonate and the like. For example, the plastic can be one used in the heath care industry, such as an iv, iv tubing, iv bag, syringe, catheter, lancet (such as those used for blood glucose testing) other device inserted into a patient.

In some examples, the prn− extract is applied to the surface and allowed to dry, thereby coating the surface.

### C. Method of Making Quercetin

In some examples, the resulting prn− extract is further treated to obtain a purified quercetin preparation. For example, the prn− extract can be applied to a column that has an affinity for quercetin. In another example, the prn− extract can be subjected to chromatography (such as TLC) to isolate or concentrate the quercetin in the extract (for example see methods in Meen and Patni, Asian J. Exp. Sci, 22:137-42, 2008; Zhou et al., J. Chromotog. 1092:216-21, 2005; Walsh et al., J. Undergrad. Chem. Res. 2:51-55, 2004).

Also provided is isolated quercetin made by the disclosed methods. In some examples, such isolated quercetin contains other components, such as plant material. In some examples, the isolated quercetin includes one or more quercetins, and other components, such as one or more of a propionic acid derivative, carbonic anhydrase, piperidine naphthalene-2-carboximida derivative, a benzoic acid derivative, inhibitor of glyoxalase, theanine derivative, di(n-acetyl-d-glucosamine, prednicarbate, coelenterazine-like compound, pyrrolo-pyrazole derivative, sulfonamide inhibitor of carbonic acid, methylsalicyluric acid, a compound, highly similar to 2S-hydroxy-10-undecanoic acid, a compound similar to aminobenzofurazan, antibiotics, a carbonic anhydrase inhibitor.

### D. Method of Increasing Tolerance of a Plant to a Stressor

The disclosure provides methods of increasing tolerance of a plant to a stressor. In some examples, the method includes functionally deleting (or genetically inactivating) one or more pirins, thus making the plant prn- for one or more genes. For example, an exogenous nucleic acid molecule that decreases pirin activity in the plant or seedling can be introduced into the plant and expressed into the plant (for example as described above), thereby increasing an amount of quercetin in the plant (such as an at least 2-fold, at least 3-fold, or at least 4-fold increase in quercetin) and increasing tolerance of the plant to one or more stressors. In some examples, expression of Prn is substantially decreased, such as a decrease of at least 50%, at least 75%, at least 90% or at least 99%. In particular examples, the method includes selecting a plant in need of increased stress tolerance. For example young seedlings of most crop plants, like soybean, are susceptible to abiotic stress in general (e.g., cold, heat, salt, high light, flooding, drought).

In another example, tolerance to a stressor is increased in a plant by a method that includes exposing the plant to a prn− extract described herein, thereby increasing the tolerance of the plant to a stressor. For example, the prn− extract can be applied to the outside of the plant (such as applied to its seeds, roots, stems, or young leaves [vegetative parts in young seedlings]), for example by spraying the prn− extract onto the plant. In another example, the plant is grown in the presence of the extract, for example in the soil in which the plant is grown, or in solution given to the plant or in which the plant is grown.

Exemplary stressors include abiotic stressor such as UV radiation (A, B or C), cold, drought, heat, and salt. Biotic stressors include hormones, fungi, bacteria, arthropods, worms, and products of biotic organisms. The disclosed methods can increase tolerance to one of these stressors, or combinations thereof. For example, in a stand of plants, prn− seedlings have a 20-50% improvement in withstanding (i.e., survival, no lodging) an abiotic stressor.

E. Method of treating a tumor

The present disclosure also provides methods for treating a tumor cell, such as a tumor present in a subject. Treatment can include reducing the number, size or volume of the tumor, decreasing growth of the tumor, decreasing metastasis of the tumor, and increasing the life span of the subject having the tumor. In some examples, a subject in need of tumor treatment, such as a subject with a tumor, suspected of having a tumor, or who has had a tumor in the past, is identified and selected for treatment.

In some examples, the method includes contacting the tumor cell with a therapeutically effective amount of an extract from prn− plants described herein, thereby treating the tumor. In some examples, for example when the tumor is in a subject to be treated, contacting the tumor includes administering a therapeutically effective amount of a prn− extract to the subject. In some examples, the prn− extract is used in combination with one or more other anti-cancer agents (such as a chemotherapeutic).

A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

In some examples, the prn− extract is used in combination with other cancer treatments, such as surgical treatment (for example surgical resection of the cancer or a portion of it) or radiotherapy, for example administration of radioactive material or energy (such as external beam therapy) to the tumor site to help eradicate the tumor or shrink it prior to surgical resection.

Exemplary tumors, such as cancers, that can be treated with the disclosed methods and prn− extracts include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T- cell malignant lymphoma). In one example, the tumor is an adenocarcinoma.

The methods and extracts can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), mixed lineage leukemia (MLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas).

### F. Method of Preventing Toxic Fungal Infection

The disclosed prn− extracts can be used as a preventative for toxic fungi, such as prevention of an infection by a toxic fungus, such as a fungus that is dependent upon phenylpropanoid concentrations of the infected cells, for example Cryptococcus gattii. Such extracts can be used to prevent or decrease the likelihood that a plant or mammalian subject will be infected with a toxic fungus. In some examples, the methods can include contacting a mammalian subject or plant with a therapeutically effective amount of a prn− extract disclosed herein, thereby decreasing or preventing a toxic fungal infection by a of the plant or mammal, such as a decrease in infection or fungal growth of at least 20%, at least 40%, at least 50%, at least 70%, at least 80%, at least 90%, or at least 95%, as compared to absence of the prn− extract.

In some examples, the prn− extract is used in combination with one or more other anti-fungal agents. For example, the subject treated with the prn− extract can also receive intravenous therapy (for 6-8 weeks or longer) with amphotericin B, either in its conventional or lipid formulation. In addition, the subject may receive oral or intravenous flucytosine. Oral fluconazole can be administered for six months or more.

In some examples, a subject or plant in need of protection from infection by a toxic fungus is identified and selected for treatment. For example, the subject can be one who is in or is expected to visit the Pacific Northwest, and who has or is expected to encounter ground soil.

The basidiomycetous yeast Cryptococcus neoformans has emerged as one of the major causative agents of meningoencephalitis in immunocompromised hosts, such as persons with AIDS, organ transplant recipients, and patients receiving high doses of corticosteroid treatment. As rates of infection have diminished in developed countries, attention is increasingly being focused on high rates of cryptococcosis in the developing countries of Africa and Asia, where cryptococcosis was found to account for an estimated 17% of AIDS-related deaths—a disease burden surpassing that of tuberculosis (French et al., Aids, 16(7):1031-8, 2002; Park et al., Aids, 23(4):525-30, 2009). Systemic infections can also occur in immunocompetent individuals; the fungus has been particularly problematic in an outbreak of Cryptococcus gattii disease on Vancouver Island in the Pacific Northwest (Stephen et al., Can Vet J, 43(10):792-4, 2002; Kronstad et al., Nat Rev Microbiol, 9(3): p. 193-203, 2011). C. gattii was formerly considered the same species as C. neoformans but is now considered a separate species based on molecular epidemiology data (Kwon-Chung et al., Toxon, 51:804-6, 2002). In addition, C. gattii displays a more severe clinical course, and is associated with an increased proclivity in macrophages driven by mitochondrial regulation (Ma et al., Proc Natl Acad Sci U S A, 106(31):12980-5, 2009).

As a basidiomycete, Cryptococcus shares phenotypes with the white rot fungi that survive in the environment by their avid ability to degrade plant matter. The white rot fungi are able to degrade lignin, a resistant plant polymer, using a destructive cocktail of laccases and lignin peroxidases (Dashtban et al., Int J Biol Sci, 5(6):578-95, 2009). In contrast, brown rot fungi, such as Postia placenta, Laetiporus portentosus and Gloeophylum trabeum, can degrade wood carbohydrates, but most do not oxidize lignin. Most ascomycetes, such as Candida albicans and Saccharomyces cerevisiae, are able to degrade cellulose and hemicellulose, but have a limited ability to degrade lignin. Notable exceptions are the agent of rice blast disease, Magnaporthe grisea, and some strains of Neurospora crassa (Martinez et al., Int Microbiol, 8(3):195-204, 2005). These relative abilities to degrade plant matter correlate with each of the fungus's environmental niches. Laccase is also a major virulence factor of Cryptococcus neoformans against humans (Salas et al.,. J Exp Med, 184(2):377-86, 1996) and its presence has been used for over 40 years as a marker of pathogenic species of Cryptococcus. Laccase in C. neoformans is expressed by two enzymes encoded by the LAC1 and LAC2 genes (Zhu and Williamson, Yeast Research, 5:1-10, 2004).

Since the first discovery of Cryptococcus in peach juice by Sanfelice in 1894 (Sanfelice, Ann d'igiene, 4:463-495, 1894) and Staib's further characterization of growth on autoclaved plant material in the early 70's, the fungus has long been associated with an ability to grow on plant matter (Staib, Zentralbl Bakteriol [Orig A], 218(4):486-95, 1971). A major reported environmental niche of the fungus is soil contaminated by pigeon guano (C. neoformans) or eucalyptus trees and decaying wood (C. gattii) (Casadevall and Perfect, Cryptococcus neoformans 1998, Wash D.C.: ASM Press.). Historically, C. gattii had been reported to share a specific ecological niche with Eucalyptus camaldulensis and E. tereticomis, which produces a nutrient-rich leaf litter and decomposing branch fall (Campisi et al., Eur J Epidemiol, 18(4):357-62, 2003; Ellis and Pfeiffer, J Clin Microbiol, 28(7):1642-4, 1990). However, examination of the outbreak on Vancouver Island has identified high concentrations of C. gattii in soils without these specific plant species. Interestingly, soil types of the tropics where C. gattii has been isolated are shared by that of Vancouver Island in the Pacific Northwest. Both are high nutrient/lignin soils by virtue of low rates of plant matter decay due to high humidity and low sunlight penetrance from the overhanging canopy, and are generally warm (above freezing temperatures) (Hattenschwiler et al., New Phytologist, 189:950-965, 2010). This indicates that lignin degradative enzymes, such as laccases produced by bacteria and fungi, play a role in the propagation of these ecological niches (Deangelis et al., PLoS One, 6(4):e19306, 2011). Recently, Cryptococcus species have been shown to mate and invade plants as an opportunistic infection through abrasions in mature plant surfaces (Xue et al., Cell Host Microbe, 1(4):263-73, 2007; Springer et al., PLoS One, 5(6):e10978, 2010). However, infection of intact plants consistent with a true plant pathogen has not been reported.

For example, the extracts can be administered to a mammal, such as applied topically to the skin or to the mucosal surface of a mammal, such as a human, mouse or other veterinary subject, thereby preventing the mammal from becoming infected with a fungus. In other example, the extracts are injected into the mammal. In some examples, the extracts are inhaled. In some examples, the subject is one who is at risk for infection by a highly toxic fungus, such as a subject who is immunocompromised, such as a subject with HIV infection or who is undergoing chemotherapy.

In some examples, the prn− extracts are contacted with plants or plant cells to prevent the plant or plant cell from becoming infected with toxic fungi. For example, as described above, the extract can be applied to the exterior of the plant, or the plant can be grown in the presence of the prn− extract. In some examples, a prn− extract is applied to a crop of plants (such as a soybean, corn, wheat, or cotton crop), thereby preventing the crop from becoming infected with a highly toxic fungus. For example, the prn− extract can be sprayed onto the plants or introduced into the water (or solution) or soil used to grow the plants.

In another example, the extracts can be applied to a surface, such as soil (such as soil in the Pacific Northwest or Vancouver Island), or a plastic surface, thereby preventing toxic fungi from growing on the surface. For example, surfaces present in a hospital, health care facility, or home could be coated with the extract (such as bed rails, counter surfaces, walls, and floors), as well as medical devices, such as catheters, iv lines, stents, and the like.

Exemplary fungi whose infection or growth can be decreased or inhibited with the disclosed prn− and prn+ extracts discussed below include, but are not limited to: Cryptococcus neoformans, Cryptococcus gattii, and other Cryptococcus species, Histoplasma capsulatum, Blastomyces dermatitidis, Coccidioides immitis, Coccidioides posadasii, Fusarium solani f. sp, Paracoccidioides brasiliensis, Penicillium marneffei, Candida species, white rot fungi (e.g., Armillaria ssp. and tinder fungus), brown rot fungi (e.g., Serpula lacrymans, Fibroporia vaillantii, Coniophora puteana, Phaeolus schweinitzii, Fomitopsis pinicola, Postia placenta, Laetiporus portentosus and Gloeophylum trabeum), Magnaporthe grisea, and Neurospora crassa.

### G. Method of Increasing Anti-Oxidant Activity

Provided herein are methods of method of increasing anti-oxidant activity in a subject. In particular examples the method includes administering to the subject a therapeutically effective amount of the prn− extract described herein, thereby increasing anti-oxidant activity in the subject. In some examples, a subject in need of increased anti-oxidant activity, such as a subject with a brain injury, is identified and selected for treatment. In another example, subjects diagnosed with cellular dysfunction diseases which put the cells under oxidative stress, inflammation and toxic waste products like Batten's disease, metabolic diseases of the lysosomes and mitochondria, are selected for treatment.

In some examples, the methods can include contacting a mammalian subject with a therapeutically effective amount of a prn− extract disclosed herein, thereby increasing antioxidant activity in the mammal, such as an increase of at least 20%, at least 40%, at least 50%, at least 70%, at least 80%, at least 90%, or at least 95%, as compared to absence of the prn− extract. In some examples, the prn− extract is used in combination with one or more other anti-oxidants.

### Extracts from prn+ Expressing Plants and Methods of Use

Transgenic plants that have increased expression or activity of pirin (Prn) have significantly decreased levels of quercetin. As a result, such plants can be used for making extracts that contain cleaved quercetin. The resulting extracts can be used for a variety of purposes, such as decreasing or preventing infection in a subject by a fungus that requires quercetin or has laccase activity and/or one to which immunocompromised individuals are susceptible, such as C. neoformans, and decreasing or preventing growth of a fungus that requires quercetin or has laccase activity and/or one to which immunocompromised individuals are susceptible, such as C. neoformans, for example growth on a surface.

A. Transgenic prn+ Plants

Transgenic plants and plant cells that are prn+ can be generated using routine methods in the art. For example, such plants and cells can include one or more exogenous nucleic acid molecules that increase pirin activity in the cell, thereby increasing an amount of functional pirin protein in the cell. Increasing pirin activity in the cell decreases levels of quercetin in the plant. Because pirin is a quercetinase, the result of increasing or activating pirin in the cell is that the resulting transgenic plants and plant cells have decreased levels of quercetin as compared to a comparable non-transgenic plant, such as at least 2 times, at least 2.5 times, at least 3 times, at least 3.5 times, or at least 4 times less quercetin.

Methods of increasing expression of a target sequence are well known in the field, and the disclosure is not limited to particular methods of increasing expression, or use of particular Prn sequences. As discussed above, Prn sequences are publicly available. In addition, recombinant methods for introducing a recombinant prn nucleic acid coding sequence into a plant or plant cell are well known. For example, a prn nucleic acid coding sequence operably linked to a promoter, for example as part of a vector, can be introduced into a plant or plant cell (for example using routine plant transformation methods, such as Agrobacterium).

Any method known in the art can be used to increase or up-regulate expression of pirin in a plant. In particular examples, a cDNA encoding a Prn protein is expressed under the control of a promoter. For example, constitutive promoters can be used to promote Prn gene expression. Constitutive promoters function under most environmental conditions. Any constitutive promoter, including variants thereof that are functionally equivalent and confer gene expression in plant tissues and cells, can be used to express a nucleic acid sequence, such as a Prn cDNA (or for example a Prn RNAi, or antisense sequence to generate a prn− plant), in a transgenic plant. Exemplary constitutive promoters include, but are not limited to, promoters from plant viruses such as the 35S promoter from CaMV (Odell et al., Nature 313:810-2, 1985; U.S. Pat. No. 5,858,742 to Fraley et al.); promoters from plant genes as rice actin (McElroy et al., Plant Cell 2:163-71, 1990); ubiquitin (Christensen et al., Plant Mol. Biol. 12: 619-32, 1989); pEMU (Last et al., Theor. Appl. Genet. 81:581-8, 1991); MAS (Velten et al., EMBO J. 3:2723-30, 1984); maize H3 histone (Lepetit et al., Mol. Gen. Genet. 231:276-85, 1992 and Atanassova et al., Plant J. 2:291-300, 1992); and the ALS promoter, a XbaI/NcoI fragment 5′ to the Brassica napus ALS3 structural gene or a nucleotide sequence with substantial sequence similarity (PCT Application No. WO 96/30530). A particular example is a rice ubiquitin gene promoter (Genbank accession no. AF184280).

In another example, the promoter used is an inducible promoter, such as a promoter responsive to environmental stimuli or synthetic chemical. Exemplary inducible promoters include those induced by heat, a chemical, or light. Use of an inducible promoter allows for controlling Prn expression.

In one example, Prn is overexpressed in a plant by using the Gateway® expression system (Invitrogen) with a native Prn promoter.

B. Generation of Extracts from prn+ Plants

The transgenic prn+ plants described above can be used for making extracts. Thus, extracts generated by such plants, referred to as prn+ extracts, are contemplated by this disclosure. In some examples, the method of making an extract includes extracting aerial portions of seedlings from transgenic plant. For example, the extracts can be generated from aerial portions of seedlings (cotyledons or cotyledons+stem) that were grown in the dark and are 7 days or less old.

In some examples, seeds of prn+ mutants are planted then grown in complete darkness. In some examples, the seeds are sterilized and rinsed in complete darkness before they are sown. In some examples, the seeds are planting in the morning (such as between 8-11 am or 9-10 am). Seeds can be maintained in the cold, for example at −0° C. to 5° C., for example 2° C. to 4° C., such as 4° C., for at least 24 hours, at least 36 hours, or at least 48 hours, such as 24 to 72 hours or 24 to 48 hours, such as 48 hours, in a sealed dark container (no light penetration). The seeds are then moved to room temperature (complete darkness), for example at least 15° C., or at least 20° C., such as 15° C. to 25° C., or 15° C. to 20° C., such as 20° C., for a period of at least 4 days, such as at least 5 days, at least 6 days, or at least 7 days, such as 4 to 8 days, 5 to 8 days, 5 to 7 days, or 5 to 6 days, such as 6 days or 7 days. In a specific example, seeds are maintained for 48 hours in 4° C. in a sealed dark container (no light penetration), then moved to 20° C. for 5 to 6 days in complete darkness.

Following growth in darkness, aerial portions of seedlings including the cotyledons are harvested, for example under a dim green light of 0.1 μmolm⁻². The aerial portions of seedlings can be harvested into a buffer, such as a buffer containing 20 mM K₂PO₄ pH 7.5 or HEPES pH 7.5, 10 mM NaCl, 1.0 mM dithiothreitol, and a 0.1% protease inhibitor cocktail for plants, at a ratio of 1 part plant material to 9 parts buffer by volume. The aerial portions are then ground up or homogenized until all material is smashed (for example for at least 1 minute, at least 3 minutes, at least 5 minutes, or at least 10 minutes, such as 1 to 5 minutes). In one example the aerial portions of seedlings are homogenized in the buffer in a bullet point glass tissue homogenizer for 5 minutes.

The resulting homogenate can then be processed to remove solid materials. For example, the homogenate can be spun at 4° C. in darkness for 5,000 rpm in a microfuge in non-extractable plastic tubes. The resulting supernatant is removed and stored in darkness at 4° C. Prior to use, the extract can be warmed, for example to room temperature, such as at least 15° C., or at least 20° C., such as 15° C. to 25° C., or 15° C. to 20° C., such as 20° C. In some examples, the extract is diluted prior to use, such as 1/20 or 1/100 volume. It was observed that the extract stayed effective when stored in the dark at 4° C., and used within 14 days.

The resulting prn+ extract can be used directly, can be concentrated, diluted in one or more pharmaceutically acceptable carriers, or combinations thereof. The pharmaceutically acceptable carriers (vehicles) useful in this disclosure are conventional and discussed above for prn− extracts. Remington's Pharmaceutical Sciences, by E. W. Martin, Mack Publishing Co., Easton, Pa., 19th Edition (1995), describes compositions and formulations suitable for pharmaceutical delivery of therapeutic compounds, such as the extracts provided herein. In particular examples, the extract is present in water or physiological saline

In some examples, the prn+ extract further includes one or more anti-fungal agents, such as a polyene antifungal (for example Natamycin, Rimocidin, Filipin, Nystatin, Amphotericin B, Candicin or Hamycin), an imidazole (for example Miconazole, Ketoconazole, Clotrimazole, Econazole, Omoconazole, Bifonazole, Butoconazole, Fenticonazole, Isoconazole, Oxiconazole, Sertaconazole, Sulconazole, Tioconazole), a thiazole (for example Fluconazole, Itraconazole, Isavuconazole, Ravuconazole, Posaconazole, Voriconazole, Terconazole), a thiazole, an allylamine (Terbinafine, Naftifine, or Butenafine), or an echinocandin. In some examples, the prn− extract further includes agents that are used to treat C. neoformans infections, such as fluconazole, Ambiosome, amphotericin B and flucytosine.

### C. Method of Preventing a Fungal Infection

The disclosed prn-+ extracts can be used as a preventative for fungi, such as prevention of an infection by a fungus that requires quercetin or has laccase activity and/or one to which immunocompromised individuals are susceptible, such as Cryptococcus neoformans. Such extracts can be used to prevent or decrease the likelihood that a plant or mammalian subject will be infected with such a fungus. In some examples, the methods can include contacting a mammalian subject or plant with a therapeutically effective amount of a prn+ extract disclosed herein, thereby decreasing or preventing a infection of the plant or mammal by a fungus that requires quercetin or has laccase activity and/or one to which immunocompromised individuals are susceptible, such as a decrease in infection or fungal growth of at least 20%, at least 40%, at least 50%, at least 70%, at least 80%, at least 90%, or at least 95%, as compared to absence of the prn+ extract. In some examples, the prn+ extract is used in combination with one or more other anti-fungal agents. In some examples, a subject or plant in need of protection from infection by such a fungus is identified and selected for treatment, such as a patient with an autoimmune disorder or an immunocompromised patient, such as those who are HIV+ or who are receiving chemotherapy.

For example, the extracts can be administered to a mammal, such as applied topically to the skin or to the mucosal surface of a mammal, such as a human, mouse or other veterinary subject, thereby preventing the mammal from becoming infected with a non-toxic fungus. In other example, the extracts are injected into the mammal. In some examples, the extracts are inhaled. In some examples, the subject is one who is at risk for infection by an opportunistic fungus, such as a subject who is immunocompromised, such as a subject with HIV infection or who is undergoing chemotherapy.

In some examples, the prn+ extracts are contacted with plants or plant cells to prevent the plant or plant cell from becoming infected with a fungus that requires quercetin or has laccase activity. For example, as described above, the extract can be applied to the exterior of the plant, or the plant can be grown in the presence of the prn+ extract. In some examples, a prn+ extract is applied to a crop of plants (such as a soybean, corn, wheat, or cotton crop), thereby preventing the crop from becoming infected with a highly toxic fungus. For example, the prn+ extract can be sprayed onto the plants or introduced into the water or soil used to grow the plants.

In another example, the extracts can be applied to a surface, such as soil or a plastic surface, thereby preventing such fungi from growing on the surface. For example, surfaces present in a hospital, health care facility, or home could be coated with the extract (such as bed rails, counter surfaces, walls, and floors), as well as medical devices, such as catheters, iv lines, stents, and the like.

Exemplary fungi whose infection or growth can be decreased or inhibited with the disclosed prn+ extracts include, but are not limited to: Cryptococcus neoformans and other Cryptococcus species, Histoplasma capsulatum, Blastomyces dermatitidis, Coccidioides immitis, Coccidioides posadasii, Paracoccidioides brasiliensis, Penicillium marneffei, Candida species, white rot fungi (e.g., Armillaria ssp. and tinder fungus), brown rot fungi (e.g., Serpula lacrymans, Fibroporia vaillantii, Coniophora puteana, Phaeolus schweinitzii, Fomitopsis pinicola, Postia placenta, Laetiporus portentosus and Gloeophylum trabeum), Magnaporthe grisea, and Neurospora crassa.

### D. Enzymatic Cleaner

The disclosed Prn+ extracts or the Prn (or Prn1) protein itself (for example in a stabilized solution) can be used as an enzymatic cleaner, for example to break down materials that may permit fungus or pathogens to survive, as many pathogens, like human cells, require antioxidants in various quantities.

Thus, as described above, Prn+ extracts or the Prn (or Prn1) protein itself can be applied to a surface to degrade proteins or other materials. In another example, Prn+ extracts or the Prn (or Prn1) protein itself can be used as a cleaner (or as part of a cleaning solution), for example to remove stains.

### Modes of Administration and Dosages

Administration is a means to provide or give a subject (or plant) an agent, such as an extract from prn− or prn+ mutant plants, by any effective route. Exemplary routes of administration to a mammalian subject include, but are not limited to, topical, injection (such as subcutaneous, intramuscular, intradermal, intraperitoneal, intratumoral, and intravenous), oral, sublingual, rectal, transdermal, intranasal, vaginal and inhalation routes. Exemplary routes of administration to a plant include, applying the agent to the plant, for example by spraying or coating the plant or a part thereof with the desired agent, or by growing the plant in the presence of the agent.

The prn− and prn+ extracts disclosed herein are administered in therapeutically effective amounts. This is an amount of a composition that alone, or together with an additional therapeutic agent(s) (such as a chemotherapeutic agent) sufficient to achieve a desired effect in a plant, subject, or in a cell, being treated with the agent. The effective amount of the agent (such as a prn+ or prn− extract) can depend on several factors, including, but not limited to the subject or cells being treated, overall health of the subject, the particular therapeutic agent, and the manner of administration of the therapeutic composition. An effective amount of an agent (such as a prn+ or prnprn − extract) can be determined by varying the dosage of the product and measuring the resulting therapeutic response, such as the regression of a tumor, prevention of a fungal infection, or increasing plant stress tolerance. Effective amounts also can be determined through various in vitro, in vivo or in situ assays. The disclosed agents (such as a prn+ or prn− extract) can be administered in a single dose, or in several doses, as needed to obtain the desired response.

In one example, a therapeutically effective amount or concentration of a prn− extract is one that is sufficient to increase the tolerance of a plant to a stressor, such as UV light. For example, a desired response can be increasing a plant's tolerance to a stressor, for example by increasing the tolerance to a particular stressor by at least 20%, at least 50%, at least 60%, at least 70%, at least 80%, at least 90%, at least 95%, at least 98%, or even at least 100%, such as at least 2-fold, at least 3-fold, at least 4-fold, at least 5-fold, or at least 10-fold, as compared to an untreated plant's tolerance to the same stressor. In another example, a desired response can be increasing a plant's tolerance to a stressor by a desired amount, for example by increasing the amount of a particular stressor that can be applied to the plant and not kill the plant, such as an increase in the dose or amount of stressor by at least 20%, at least 50%, at least 60%, at least 70%, at least 80%, at least 90%, at least 95%, at least 98%, or even at least 100%, such as at least 2-fold, at least 3-fold, at least 4-fold, at least 5-fold, or at least 10-fold.

In one example, a therapeutically effective amount or concentration of a prn− extract is one that is sufficient to treat a tumor, for example by preventing advancement (such as metastasis), delay progression of the tumor, or to cause regression of the tumor, or which is capable of reducing symptoms caused by the tumor. In one example, a therapeutically effective amount or concentration is one that is sufficient to increase the survival time of a patient with a tumor. The treatment does not have to be completely effective (e.g., the tumor need not be completely eliminated) for the composition to be effective. For example, administration of a composition containing a prn− extract can decrease the size of a tumor (such as the volume or weight of a tumor, or metastasis of a tumor), or metastasis of a tumor for example by at least 20%, at least 50%, at least 80%, at least 90%, at least 95%, at least 98%, or even at least 100%, as compared to the tumor size in the absence of the prn− extract. In one particular example, a desired response is to kill a population of tumor cells, for example by killing at least 20%, at least 50%, at least 60%, at least 70%, at least 80%, at least 90%, at least 95%, at least 98%, or even at least 100% of the tumor cells, as compared to the cell killing in the absence of the prn− extract. In one particular example, a desired response is to increase the survival time of a patient with a tumor (or who has had a tumor recently removed) by a desired amount, for example increase survival by at least 20%, at least 50%, at least 60%, at least 70%, at least 80%, at least 90%, at least 95%, at least 98%, or even at least 100%, as compared to the survival time in the absence of the prn− extract.

In one example, a therapeutically effective amount or concentration of a prn− extract is one that is sufficient to prevent infection by a toxic fungus, such as C. gattii. In one example, a therapeutically effective amount or concentration is one that is sufficient to decrease infection of a patient by a toxic fungus, such as C. gattii. For example, administration of a composition containing a prn− extract can decrease the likelihood that a patient will become infected by a toxic fungus, such as C. gattii, for example a decrease of at least 20%, at least 50%, at least 80%, at least 90%, at least 95%, at least 98%, or even at least 100%, as compared to the rate of infection in the absence of the prn− extract.

In one example, a therapeutically effective amount or concentration of a prn− extract is one that is sufficient to prevent growth of a toxic fungus, such as C. gattii, for example growth on a surface. For example, contact of a toxic fungus, such as C. gattii, with a composition containing a prn− extract can decrease the growth of the fungus, for example a decrease of at least 20%, at least 50%, at least 80%, at least 90%, at least 95%, at least 98%, or even at least 100%, as compared to the growth in the absence of the prn− extract.

In one example, a therapeutically effective amount or concentration of a prn− extract is one that is sufficient to increase anti-oxidant activity, for example in a subject. For example, a desired response can be increasing anti-oxidant activity, for example in a subject, for example by increasing the anti-oxidant activity by at least 20%, at least 50%, at least 60%, at least 70%, at least 80%, at least 90%, at least 95%, at least 98%, or even at least 100%, such as at least 2-fold, at least 3-fold, at least 4-fold, at least 5-fold, or at least 10-fold, as compared to anti-oxidant activity in the absence of the prn− extract.

In one example, a therapeutically effective amount or concentration of a prn+ extract is one that is sufficient to prevent infection by a fungus that requires quercetin or has laccase activity, such as C. neoformans. In one example, a therapeutically effective amount or concentration is one that is sufficient to decrease infection of a patient by a fungus that requires quercetin or has laccase activity, such as C. neoformans. For example, administration of a composition containing a prn+ extract can decrease the likelihood that a patient will become infected by such a fungus, such as C. neoformans, for example a decrease of at least 20%, at least 50%, at least 80%, at least 90%, at least 95%, at least 98%, or even at least 100%, as compared to the rate of infection in the absence of the prn+ extract.

In one example, a therapeutically effective amount or concentration of a prn+ extract is one that is sufficient to prevent growth of a fungus that requires quercetin or has laccase activity, such as C. neoformans, for example growth on a surface. For example, contact of a fungus that requires quercetin or has laccase activity, such as C. neoformans, with a composition containing a prn+ extract can decrease the growth of the fungus, for example a decrease of at least 20%, at least 50%, at least 80%, at least 90%, at least 95%, at least 98%, or even at least 100%, as compared to the growth in the absence of the prn+ extract.

In particular examples, a therapeutically effective dose of a prn+ or prn− extract for administration to a subject, such as a human or veterinary subject, is at least 0.5 milligram per 60 kilogram (mg/kg), at least 5 mg/60 kg, at least 10 mg/60 kg, at least 20 mg/60 kg, at least 30 mg/60 kg, at least 50 mg/60 kg, for example 0.5 to 50 mg/60 kg, such as a dose of 1 mg/60 kg, 2 mg/60 kg, 5 mg/60 kg, 20 mg/60 kg, or 50 mg/60 kg, for example when administered iv. In another example, a therapeutically effective dose of a prn+ or prn− extract is at least 10 μg/kg, such as at least 100 μg/kg, at least 500 μg/kg, or at least 500 μg/kg, for example 10 μg/kg to 1000 μg/kg, such as a dose of 100 μg/kg, 250 μg/kg, about 500 μg/kg, 750 μg/kg, or 1000 μg/kg, for example when administered intratumorally or ip. In one example, a therapeutically effective dose of a prn+ or prn− extract is at least 1 μg/ml, such as at least 500 μg/ml, such as between 20 μg/ml to 100 μg/ml, such as 10 μg/ml, 20 μg/ml, 30 μg/ml, 40 μg/ml, 50 μg/ml, 60 μg/ml, 70 μg/ml, 80 μg/ml, 90 μg/ml or 100 μg/ml administered in topical solution. However, one skilled in the art will recognize that higher or lower dosages also could be used, for example depending on the particular prn+ or prn− extract.

In one example, the method includes administration or application of at least 1 μg of a prn+ or prn− extract to the plant, surface or subject (such as a human subject). For example, at least 1 μg or at least 1 mg of the prn+ or prn− extract can be administered daily, such as 10 μg to 100 μg daily, 100 μg to 1000 μg daily, for example 10 μg daily, 100 μg daily, or 1000 μg daily. In one example, the subject is administered at least 1 μg (such as 1-100 μg) intravenously of the prn+ or prn' extract. In one example, the subject is administered at least 1 mg intramuscularly (for example in an extremity) of such prn+ or prn− extract. The dosage can be administered in divided doses (such as 2, 3, or 4 divided doses per day), or in a single dosage daily.

In particular examples, such daily dosages are administered in one or more divided doses (such as 2, 3, or 4 doses) or in a single formulation. In particular examples, the plant subject is administered the prn+ or prn− extract on a multiple daily dosing schedule, such as at least two consecutive days, 10 consecutive days, and so forth, for example for a period of weeks, months, or years. In one example, the subject is administered prn+ or prn− extract daily for a period of at least 30 days, such as at least 2 months, at least 4 months, at least 6 months, at least 12 months, at least 24 months, or at least 36 months.

The disclosed prn+ and prn− extracts can be administered alone, in the presence of a pharmaceutically acceptable carrier, in the presence of other therapeutic agents (such as other anti-neoplastic agents).

### Example 1

### Functional Deletion of Pirin in Plants Increases Stress Tolerance

This example describes methods used to decrease pirin expression in plants, and shows the effect of decreased pirin expression on tolerance to UV stress.

### Materials and Methods

Plant Materials and Accessions.

Matched seed lots of wild type (wt) Columbia (col) Arabidopsis and a mutant carrying a T-DNA insertion within coding region of PRN1 (SALK_{—}006939) were obtained from the Arabidopsis Biological Resource Center (Columbus, Ohio; Alonso et al., Science, 301(5633):653-7, 2003). The Pirin1 mutant line was homozygous for the reported insertion. Plants intended for seed stocks were grown in Scott Metromix 200 (Scotts; Marysville, Ohio) in continuous white light as described elsewhere (Lapik and Kaufman, Plant Cell. 15(7):1578-90, 2003). Gene sequence accessions were obtained from GenBank and SIGnAL (both available on the internet) and compared by CLUSTALX program.

Plant Growth Conditions.

Seedlings of Arabidopsis thaliana wt col or insertion mutants were grown on 0.8% agarose plates containing only 0.5× Murashige and Skoog media as described in Lapik and Kaufman (Plant Cell. 15(7):1578-90, 2003). The growth media contained no additional sugar, hormones, vitamins or other nutrients. Seedlings were grown for 6-7 days in complete darkness, then exposed (or not) to an abiotic signal. 24 hours later, aerial portions were harvested to measure the pirin1 activity, amino acid analysis, in vitro translation and quercetin activity. RNA analysis is described in Lapik and Kaufman (Plant Cell. 15(7):1578-90, 2003). Low fluence UV irradiations were conducted as described previously (Warpeha et al., Plant. Cell and Envir. 31:1756-70, 2008). Briefly, 100 seedlings were planted. Etiolated seedlings were treated of day 6 with 254 nm. 24 hours later, plants were photographed in white light from the side angle or harvested.

Generation of Plant Extracts.

Seeds of null prn1 Arabidopsis mutants were sterilized, rinsed then sown in complete darkness. Seeds were maintained for 48 h in 4° C. in a sealed dark container (no light penetration), then moved to 20° C. for 6 days in complete darkness. On day 6, seedlings were given a total dose of 10⁴ μmolm⁻² of 317 nm or 10⁴ μmolm⁻² of other wavelengths of UV with no other irradiation and immediately returned to complete darkness. 24 h later aerial portions of seedlings under a dim green light of 0.1 μmolm⁻² were harvested into a buffer (20 mM K₂PO₄ or HEPES pH 7.5, 10 mM NaCl, 1.0 mM dithiothreitol, 0.1% protease inhibitor cocktail for plants) then ground in a bullet point glass tissue homogenizer for 5 minutes until all material was smashed. The homogenate was spun at 4° C. in darkness for 5,000 rpm in a microfuge in non-extractable plastic tubes. Supernatant was removed by aspiration and stored in darkness at 4° C. Extract was stored in darkness until just prior to use, then warmed to 20° C. prior to application in 1/20 or 1/100 volume to volume of culture.

Absorbance Spectra.

Acetone dried samples were mixed with disodium hydrogen phosphate (pH 7.4) buffer and filtered once using a 0.45 μm syringe filter. Absorption spectra between 200 to 450 nm were obtained using a duel beam spectrophotometer (Spectronic Genesys 5, Thermo Electron Corporation; Madison, Wis.).

Quercetin Activity Assays.

Full-length PIRIN1 template was prepared, amplified and purified by the methods described (Lapik and Kaufman, Plant Cell. 15(7):1578-90, 2003; Warpeha et al., Plant Physiol. 140:844-55, 2006). Pirin1 proteins were individually produced by coupled in vitro transcription/translation using TNT T7 Coupled Wheat Germ Extract System (Promega; Madison, Wis.) as directed and as modified previously (Warpeha et al., Plant Physiol. 140:844-55, 2006). Translated Pirin1 was subsequently incubated with quercetin in complete darkness in a 26° C. circulating water bath for 15 minutes.

The quercetin 2,3-dioxygenase activity of Pirin1 was determined as described in Adams and Jai (J. Biol. Chem. 280(31):28675-82, 2005). Briefly, the quercetin 2,3-dioxygenase activity of pirin wase assayed at 295 K for 5 min in a reaction mixture containing 50 mm NaH₂PO₄, pH 8.0, 300 mm NaCl, 60 μm quercetin in Me₂SO, and 18.5 nm enzyme. Activity was observed by following the decrease in the absorbance maximum for quercetin, which occurs at 384 nm at pH 8.0. The inhibitors kojic acid, sodium diethyldithiocarbamate, and 1,10-phenanthroline monohydrochloride were dissolved in Me₂SO and added to the reactions at a final concentration of 50 nm.

In Vitro AtGPA1-AtPirin1 Activity Assay.

Translated Pirin1 was prepared as described above and in vitro association assays were conducted in assay buffer (pre-incubations included magnesium) at 26° C. (Adams and Jai, J. Biol. Chem. 280(31):28675-82, 2005; Warpeha et al., Plant Physiol. 140:844-55, 2006). GPA1 was incubated with Pirin1 in a 1:1 ratio. “Activated” Pirin1 was achieved by pre-incubation with 100 μM GTPγS (a non-hydrolysable GTP analog). “Inactivated” Pirin1 was achieved by pre-incubation with GDP, or GDPγS (a non-hydrolysable GDP analog). Both pre-incubations occurred overnight in darkness at 10° C.

HPLC Preparation and Spectral Analysis.

Structure analysis was conducted as described (Razal et al., Phytochem. 41:31-35, 1996; Razal et al., Phytochem. Analysis; 5:98-104, 1994) with modifications in Warpeha et al., Plant Physiol. 140:844-55, 2006. Quercetin and kaempferol content was assessed in the aerial portions of wt col and the insertion mutant seedling (Pirin1) 4 h after UV-irradiation treatment. Seedlings were grown and irradiated as described above, aerial portions harvested in liquid nitrogen, ground to a fine powder, and stored at −80° C. until compound analysis could be performed. All procedures hereafter were conducted at 4° C. or on ice unless specified.

Microscope Images.

Fluorescent images were obtained of living unfixed seedlings 24 h post UV treatment. Optical sectioning was achieved by using a Zeiss Axiovert 200M microscope (Carl Zeiss; Oberkocken, Germany), equipped with ApoTome (collected by grid projection) and a real color digital camera and the DAPI-Long Pass filter set (Chroma; Rockingham, Vt.) in order to collect all UV and visible wavelengths. Photographs of whole cotyledon fluorescence were snapped on the same microscope set up, minus the apoTome setting.

Kill Assay.

An assay of survival of UV-C radiation was performed as detailed in (Warpeha et al., Plant. Cell and Envir. 31:1756-70, 2008).

### Results

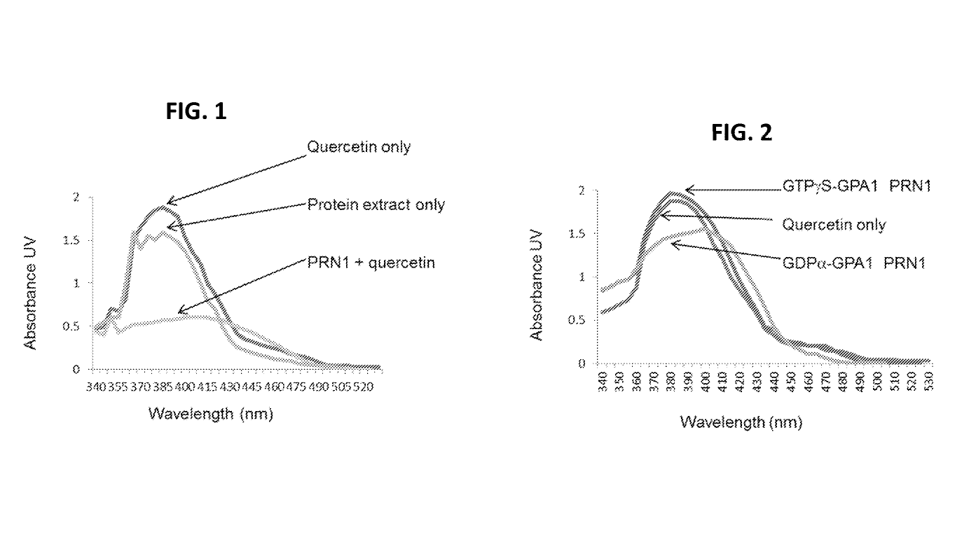

A. thaliana Pirin1 (AtPirin1) was analyzed for its' ability to cleave quercetin. As shown in FIG. 1, in vitro-translated AtPirin1 results in specific cleavage of quercetin. Quercetin is expected to have a normal absorbance maximum of 384 nm and this was observed. However, upon the addition of AtPirin1, this value was shifted to −405-410 nm within 15 min at 26° C. The protein extract itself, which contains all the components of translation to produce AtPirin1, did not cause a shift in the absorbance spectrum peak and comparatively did not cause major change to the quercetin absorbance. Thus, the observed quercetinase activity of AtPirin1 was not due to the in vitro translation protein extract itself which contains all components of translation and assay except PRN1.

The observed AtPirin1 quercetinase activity is regulated through its' interactions with GPA. In vitro-translated AtGPA1 (a-subunit of the single-copy G-protein in Arabidopsis) was pre-incubated with GTPγS (a non-hydrolyzable GTP analog) overnight to bind the AtGPA1 to keep the protein in the permanently activated conformation, or AtGPA1 was pre-incubated with GDPαS (a non-hydrolysable GDP analog) to keep the protein in the permanently inactive conformation in order to determine in vitro which G-protein conformation AtPirin1 associates with, if at all, in order to carry out quercetinase activity. AtGPA1 bound to one of the two non-hydrolyzable analogs was then incubated with AtPirin1 and quercetin in conditions identical to that for FIG. 1. The absorbance profile of the reactions indicated that AtPirin1 had no quercetinase activity if associated with activated conformation of AtGPA1, displaying the same absorbance spectrum as quercetin alone (FIG. 2). However, the inactivated conformation of AtGPA1 associated with AtPirin1 to permit quercetinase activity, indicated by the absorbance spectrum, with the peak shifted to 405-410 nm (FIG. 2). The quercetinase activity of PRN1 is off when GPA1 is in its active conformation and on when GPA1 is in an inactive conformation. This indicates that activation of the stress-responsive GCR1-GPA1 pathway leads to inactivation of PRN1 and increased levels of quercetin.

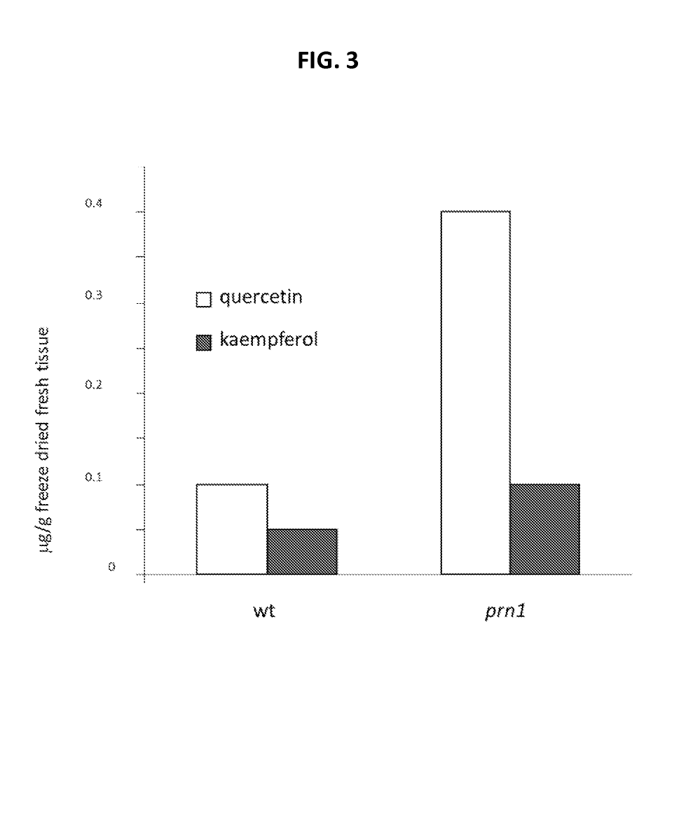

To confirm that AtPirin1 protein was relevant as a quercetinase, T-DNA insertion null mutants of AtPirin1 were used, where throughout the plant there is no AtPirin1 RNA or protein detected. HPLC analysis of etiolated seedlings was performed on AtPirin1 mutant seedlings to measure the levels of extractable quercetins and the closely related compound kaempferol. As shown in FIG. 3, elimination of the PRN1 gene leads to high levels of quercetin in etiolated seedlings. Quercetin levels in prn1 mutants are about four times higher than in wild type. Conversely, levels of closely related compounds, such as kaempferol remained unchanged (not significant) between AtPirin1 mutants and wild type plants.

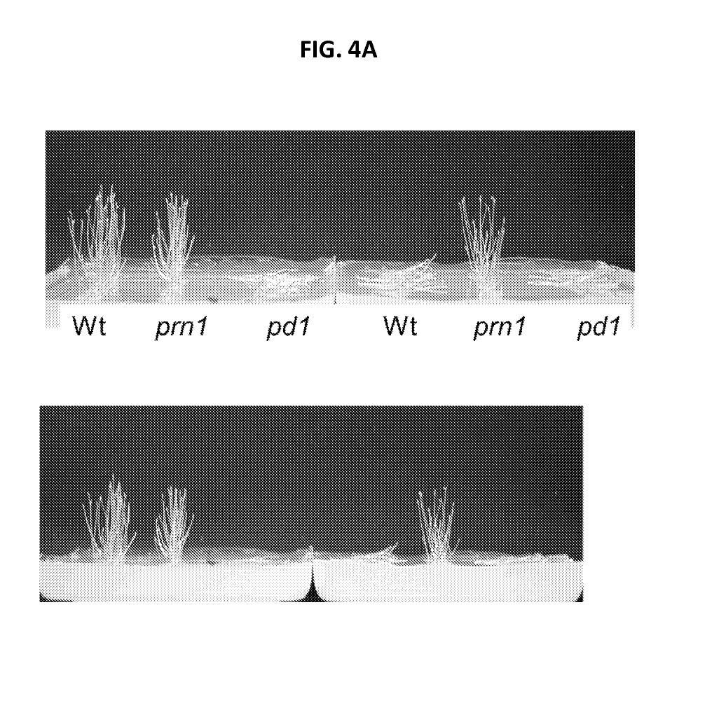

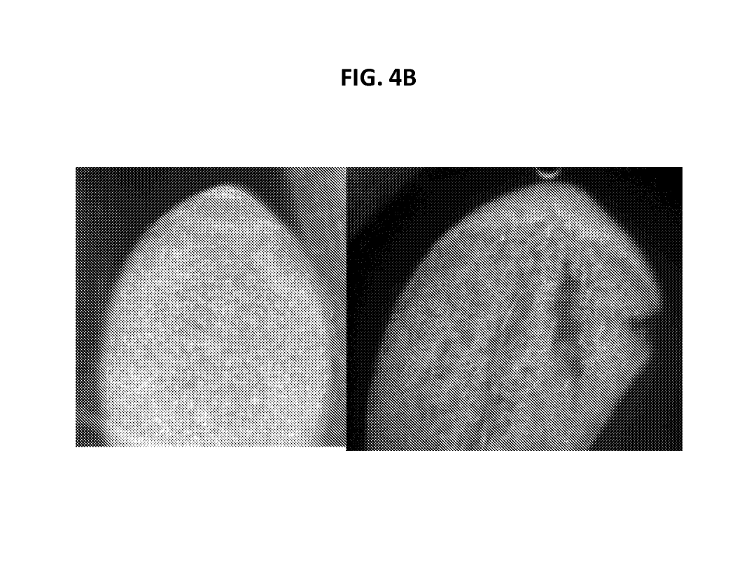

To demonstrate that elevated quercetin due to the lack of AtPirin1 in young etiolated seedlings alters the ability of the plant to respond to stressful stimuli, genetic mutants were used. As shown in FIG. 4A, even low dose UV-C radiation (10⁵ μM m⁻² UVC radiation wild types dies; wild type can only survive 10⁴ max) can cause damage and death to some genotypes, and at higher radiation levels, AtPirin1 mutants survive radiation better than wild type plants. FIG. 4A demonstrates that small amounts of 254 nm (4 min treatment; left panel) do not kill wild type plants or prn1 mutants, but even 8 min of 254 nm causes lodging of the wild type whole plants (right panel). In contrast, prn1 mutants survive. Upon microscopic evaluation, major cell damage was observed in wild type plants, but not in prn1 mutants As shown in FIG. 4B, prn1 mutants make an excess of pigments/light absorbing compounds. Thus, quercetin is protects plants from potential DNA-damaging radiation.

### Discussion

Plants from the time they germinate from seed must integrate a number of external environmental signals occurring simultaneously. They also must alternate and/or regulate certain activities that occur only during the day versus activities that occur at night (in darkness). How they respond to environmental signals in the transition from seed to young growing plant is complex as the seeds only have a small store of materials to support the new plant until it is photosynthetically fully capable. One of the key compounds stored in seeds of many types is quercetin, a potent antioxidant and structure capable of absorbing UV-radiation.

Pirin and Quercetin have Multifaceted Role in Plants.

AtPirin1 (Pirin 1), one of five Pirin orthologs identified in Arabidopsis thaliana (Arabidopsis) (Hihara et al., FEBS Lett. 574(1-3):101-5, 2004), is expressed in young (6-7 day old) etiolated Arabidopsis and functions in the stress-responsive GCR1-GPA1-PD1/ADT3 G-protein-mediated signal transduction pathway as a GCR1-GPA1 effector. AtPirin1 has a specific interaction with GPA1 and acts as the G effector in the signaling mechanism that inhibits the ABA-mediated delay in germination (Lapik and Kaufman, Plant Cell. 15(7):1578-90, 2003). AtPirin1 was originally identified through its interactions with NF-Y, the heterotrimeric CCAAT box binding proteins (Wendler et al., J. Biol. Chem. 272(13):8482-9, 1997), indicating a role for NF-Y in the GPA1-mediated interference in the ABA-induced delay of germination, and a potential link between GPA1 and Lhcb expression, regulated by abscisic acid- and blue-light-mediated gene expression via its interaction with NFY (A5,B9,C4) and the CCAAT box located in several ABA- and BL-responsive genes (Warpeha et al., Plant Physiol. 144(4):1590-1600, 2007; Warpeha et al., Plant Physiol. 140:844-55, 2006). The abiotic signal-responsive G-protein pathway is a rapid-response system, responsible not only for gene expression, but also for the enhanced production of Phenylalanine via the PD1/ADT3 protein, thereby, the stress-protective phenylpropanoid compounds which include quercetin (Warpeha et al., Plant Physiol. 140:844-55, 2006). Quercetin is among the most abundant of the phenylpropanoids synthesized under stress or pre-stress conditions. In addition to being an efficient absorber of UV-B, quercetin also exhibits strong anti-oxidant capabilities, indicting it can assist in the prevention of damaging effects of many different types of abiotic and biotic stress. Complementing this idea, quercetin was recently found to bind the ER stress-induced kinase-endonuclease IRE1 to function alongside stress signals from the ER lumen to modulate IRE1. Little is known, however, about how quercetin levels are regulated in any cell type, but it is reported that concentrations above 20 μM in animal cells are considered toxic.

Quercetinase Activity in Plant Cells is in Association with the Inactivated Form of G-protein α Subunit.

Recently, pirin protein was found to possess enzymatic activity, with roles as a quercetinase in both bacteria and humans (Adams and Jai, J. Biol. Chem. 280(31):28675-82, 2005), cleaving quercetin to carbon monoxide and 2-protocatechuoylphloroglucinol carboxylic acid (Oka and Simpson, Biochem Biophys Res Commun. 43(1):1-5, 1971). The AtPirin1 protein did not have any enzymatic activity on quercetin if AtGPA1 was in the active conformation—in fact quercetinase activity was completely prevented compared to the test of activity where AtPirin1 was added directly to quercetin with no other proteins (Adams and Jai, J. Biol. Chem. 280(31):28675-82, 2005). This is interesting as the active conformation of AtGPA1 is critical for transcription, providing a separation of activities based on Gα conformation.

The assay with in-vitro translated AtGPA1 and AtPirin1 led to less quercetinase activity indicating that other G-protein components or effector molecules are typically associated with AtPirin1. Furthermore, it is also possible that AtPirin1 may typically be in repression in the plant cell to avoid cleaving too much antioxidant, where it was clear that AtPirin alone cleaved quercetin rapidly and effectively. This is an intriguing concept as many studies of cancer cells indicate the dysregulaton of pirin is key to the cellular pathology, and has been shown to cause detrimental effects to the cell cycle (Licciulli et al., Leukemia. 24(2):429-37, 2010). Quercetin has been considered a potent antioxidant that may be a useful treatment for the malignancy. Hence, pirin may be an important regulator in cell stress in response to environmental changes.

AtPirin Mutants Demonstrate an Increase in Quercetin, and an Increased Resistance to UV Radiation.

These findings indicated that elimination of the AtPirin1 protein in young seedlings would lead to elevated levels of quercetin in etiolated seedlings. HPLC analysis of etiolated seedlings confirms that quercetin levels in Pirin1 mutants are four times higher than in wild type, while levels of the closely related compounds are unchanged and there is a corresponding increase in natural fluorescence, correlating with survival of AtPirin1 mutant seedlings after treatment with apoptosis-capable levels of UV-C. Lower levels of antioxidants and higher levels of pirin or dysregulated pirin could have a significant impact on a mammalian cell's ability to survive cellular stress and mutations from UV and other radiations and chemical stressors.

Model for Cellular Antioxidant (Quercetin) Regulation.

From AtPirin1's activities in the young plant the following model is proposed (FIGS. 5A and 5B). As shown in FIG. 5B, when an abiotic signal (e.g., salt, heat, UV-B) is perceived by the seedling, which leads to the activation of GCR1 then GPA1, activated GPA1 binds PD1/ADT3, leading to synthesis of phenylalanine. The increase in phenylalanine then leads to an increased synthesis and/or deployment of specific compounds, such as quercetin, to the young developing leaves of the seedling. GPA1 can simultaneously interact with Pirin1 leading to the turning off of the Pirin1 quercetinase activity and allowing Pirin1 to interact with NFY and affect gene expression. As shown in FIG. 5A, in the absence of stimulation by an abiotic signal, PD1/ADT3 is functioning at a low level of activity, synthesizing only small amounts of phenylalanine and therefore producing only low levels of quercetin. In this situation, lack of stimulation of the G-protein α-subunit, AtPirin1 functions as a quercetinase, breaking down accumulated quercetin in order to keep levels below toxic levels, and fewer stressors are experienced overnight (i.e., less heat-shock, no UV, no white light) so less quercetin is needed.

### Example 2

Extracts from prn1 Mutant Plants Kill Cancer Cells

This example describes experiments used to show that extracts generated from prn1 mutant plants can kill cancer cells in vitro.

The MCF-7 breast adenocarcinoma cell line and MCF-10a non-tumorigenic breast epithelial cell line were used. Cells were grown under sterile conditions. Cells were trypsinized to remove them from the flasks. Cells were washed and resuspended in 2 mL media then counted. Cells were diluted in media to 5×10⁴ cells/mL and grown overnight at 37° C.

Extracts from the prn1, pd1 or wt plants was generated as described in Example 1, UV-B irradiated, then stored in darkness until just prior to use, and warmed to 20° C. prior to application in 1/20 or 1/100 volume to volume of culture. Effectiveness of extract was determined by treating normal, non-invasive cancer and invasive cancer cell lines with either the prn1, pd1/adt3 or wt extract then assessed over the next few days.

Cells were then treated with 100 μl of (1) PBS; (2) wild-type extract; (3) PD1 extract; (4) PRN (prn− extract); (5) extract from PRN+PD1+WT (33 μl each); or (6) no treatment. Cells were then incubated for 48 hours.

Cells were stained with calcein AM (2 ml of a 4 μM calcein AM solution) and imaged with a FITC filter set at 20× to identify live cells. Cell coverage area was counted with a metamorph integrated morphometry analysis. The data were normalized to the untreated samples used in the same experiment.

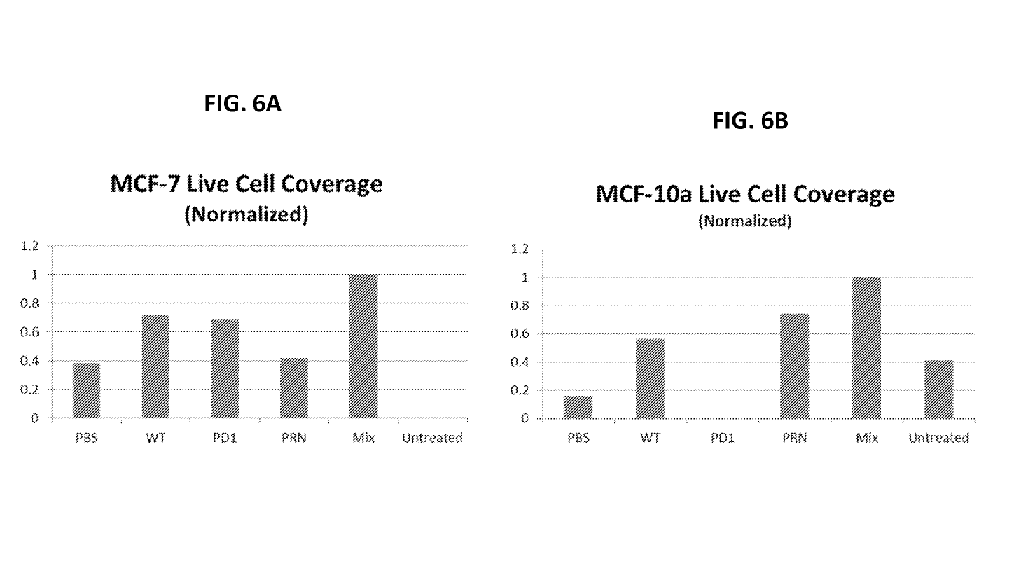

As shown in FIG. 6A, treatment of MCF-7 cancer cells with the PRN extract reduced growth of the cancer cells. In contrast, treatment of MCF-10a non-cancer cells with PRN extract had no effect on cell growth (FIG. 6B).

Cells were also stained with DAPI. Cells were in incubated for 12 minutes with 1 ug/mL DAPI in PBS w/ (37° C.), and cells imaged with a DAPI filter at 20×. Cells treated with prn1-extract killed cells more quickly than any other treatment.

The media color was also monitored. Yellow color indicates more acidic media, which usually means cells are beginning to die and lyse their contents into the media, while pink color indicates less acidic media and cell growth.

### Example 3

### Susceptibility of Intact Germinating Arabidopsis thaliana to Cryptococcus

This example describes methods used to demonstrate that prn1 mutant plants were resistant to Cryptococcus gattii than plants expressing wild-type levels of pirin. This example investigated inoculation of seeds of Arabidopsis thaliana with fungal cells over a 21-day period of dim light, simulating that encountered on a forest floor, or bright light, simulating open fields. C. gattii was the more virulent plant pathogen, resulting in disrupted germination as well as increased stem lodging, fungal burden and plant tissue co-localization. C. neoformans was a less virulent plant pathogen, but also produced significant rates of stem lodging, fungal burdens and tissue residence, and was equally successful in high light exposure. Arabidopsis mutants of the GPA1 pathway, a stress-related signal transduction pathway, showed altered susceptibility to cryptoccocal infections, indicating roles for this pathway in plant defense. Laccase, a fungal virulence factor against humans, was also implicated in plant pathogenesis, as cryptococcal lac1Δ strains were less virulent than wild-type cells. These results are the first to demonstrate the pathogenic capacity of cryptococcal species against healthy plants under physiologically relevant conditions.

To understand the role of Cryptococcus as a plant pathogen, the model plant, Arabidopsis thaliana was used. Since Cryptococcus spp. are found predominantly in soils and rotting vegetation (Dimenna, J Gen Microbiol, 11(2):195-7, 1954; Emmons, J Bacteriol, 62(6):685-90, 1951), to model physiological pathogenesis within the context of the leaf litter/forest floor, plants were inoculated during their germination period in dim light conditions. It was observed that Cryptococcus neoformans and, to a greater extent, C. gattii were both able to grow on and infect germinating intact seedlings during colonization of the plant surface. Interestingly, defects in two GPA1 (Gα subunit) effectors, PRN1 and PD1/ADT3, resulted in increased susceptibility to infection by C. neoformans, but an increased resistance to infection by C. gattii as determined by both seedling survival studies as well as fungal burden recovered from seedlings. This indicates that the more successful plant pathogen, C. gattii has evolved specific mechanisms to exploit stress responses induced by this GPA1 plant pathway. In addition, a C. neoformans lac1Δ strain, unable to process phenolic substrates, demonstrated a reduced ability to kill intact germinating seedlings, establishing laccase as a fungal virulence factor in plant as well as animal pathogenesis. These observations extend the ecological niche of Cryptococcus to include intact germinating seedlings, and indicate that a role in phenolic utilization during plant pathogenesis may have contributed to the optimization of the laccase virulence trait during evolution of the pathogen.

### Materials & Methods

Fungal Strains, Plasmids and Media.

Cryptococcus neoformans ATCC 208821 (H99) was a gift of J. Perfect and the R265 strain of C. gattii was a gift of J. Heitman. All strains were grown on YPD medium (1% yeast extract, 2% Bacto peptone, and 2% dextrose). Solid media contained 2% Bacto agar.

Accessions of Seeds & Plant Growth Protocol.

Matched seed lots of wt Columbia (col) Arabidopsis thaliana and seedling accessions carrying T-DNA insertions within coding regions of PD1/ADT3 (SALK_{—}029949) and Pirin1 PRN1 (SALK_{—}006693) [Warpeha et al., Plant Physiol., 140(3):844-55, 2006; Warpeha et al., Plant Physiol., 143(4):1590-600, 2007] were obtained from the Arabidopsis Biological Resource Center (Columbus, Ohio, USA) (Alonso et al., Science, 301(5633):653-7, 2003). Gene sequence accessions were obtained from Genbank (www.ncbi.nlm.nih.gov) and SIGnAL (signal.salk.edu). Seeds were surface-sterilized and planted in 0.8% top agarose stabilized with 0.5× Murashige and Skoog (MS)/2-(N-morpholino)ethanesulfonic acid (MES) pH 5.8 minimal media for plant growth (50-120 seeds per disk depending on experiment), on 0.5×MS/MES pH 5.8 plates (50 ml of media/agarose in a phytatray). Planted seeds were subjected to a cold treatment (4° C.) for 48 h to stimulate synchronized germination (Warpeha et al., Plant Physiol., 91(3):1030-5, 1989) but no light vernalization/treatment was performed. Sets of planted seeds after the 48 h cold period (vernalization) were grown in complete darkness for 48 h at 20° C. then incubated for the indicated periods in either dim light (0.1 μmol m⁻² s⁻¹ for 16 h cycled with complete darkness for 8 h during each 24 h period), or full summer day light (10 μmol m⁻² s⁻¹ for 16 h followed by complete darkness for 8 h). All seedlings were maintained at 20° C. for the indicated growth periods with sufficient moisture to avoid water stress. During dark incubation periods, planting and inoculation of germinating seeds were performed under a dim green safelight.

Inoculation with Cryptococcus.

Seeds were inoculated with Cryptococcus culture of the indicated strains at a time after removal from the 4° C. treatment, prior to placement in the 20° C. incubator. Cryptococcus strains were grown on plates, 1 colony was selected and dispersed in 0.5×MS/MES media pH 5.8, then read on a spectrophotometer for cell density. Culture was diluted to 0.2 optimal density (OD) at 595 nm. The culture at 0.2 OD was diluted 1:10 vol/vol and 200 μl was applied evenly to the phytatray (9.5×8 cm) on top of the sown seeds (which were in a disk of −1 mm thick on top of the plate) so there was an even layer of Cryptococcus culture at a low concentration. After liquid culture applied to the phytatray had soaked in the plates (5 min), the plates were maintained under growth protocol and observed over time.

Observation and Experimental Assessment.

Growth characteristics were observed 7, 14, 21 and 28 d after vernalization. At the indicated time points, sets of seedlings were observed under a dissecting microscope for morphological changes including successful seed germination, growth and stem lodging (defined when seedlings fall over due to significant stress/damage which typically is not recoverable, indicating death of the plant), then sacrificed for fungal burdens. Seedlings were assessed for fungal burdens by harvesting aerial portions, (50 seedlings) followed by homogenization in 1 ml in MS/MES media pH 5.8 at 4° C. and inoculation of aliquots onto YPD agar followed by 20° C. up to 7 days, whereby colony formation units (CFU) were assessed.

Statistics:

Statistical significance of seedling survival times was assessed by Kruskall-Wallis analysis (ANOVA on Ranks). Statistical analysis was conducted using GraphPad Prism software, version 4.03. In mouse mortality studies, time of death of the survivors was recorded as the day of experimental termination. Survival of seedlings at 21 days was performed by Chi Square using a contingency table and fungal burden compared by t-test of experiments conducted in triplicate.

Results:

Co-Incubation of Cryptococcus neoformans with Seeds from Arabidopsis thaliana Leads to Significant Stem Lodging During Early Growth.

To simulate an environment in which plant and fungal communities co-exist, we planted seeds of Arabidopsis thaliana (120 seeds per incubation), sowed a suspension of fungal cells of Cryptococcus in the plant medium at the same time and observed for effects on germination and growth of seedlings at 14 days and 21 days. Seedlings were incubated under either dim light, to simulate conditions under an overhead canopy, or brighter light, to simulate sunlight in open spaces. Because of C. neoformans's global distribution, the first fungal strain studied was a C. neoformans serotype A strain H99, first isolated from a patient with Hodgkin's disease having meningoencephalitis (Perfect et al., Am J Pathol, 101(1):177-94, 1980). Seedling viability was measured by a loss of stem integrity also known as ‘stem lodging’.

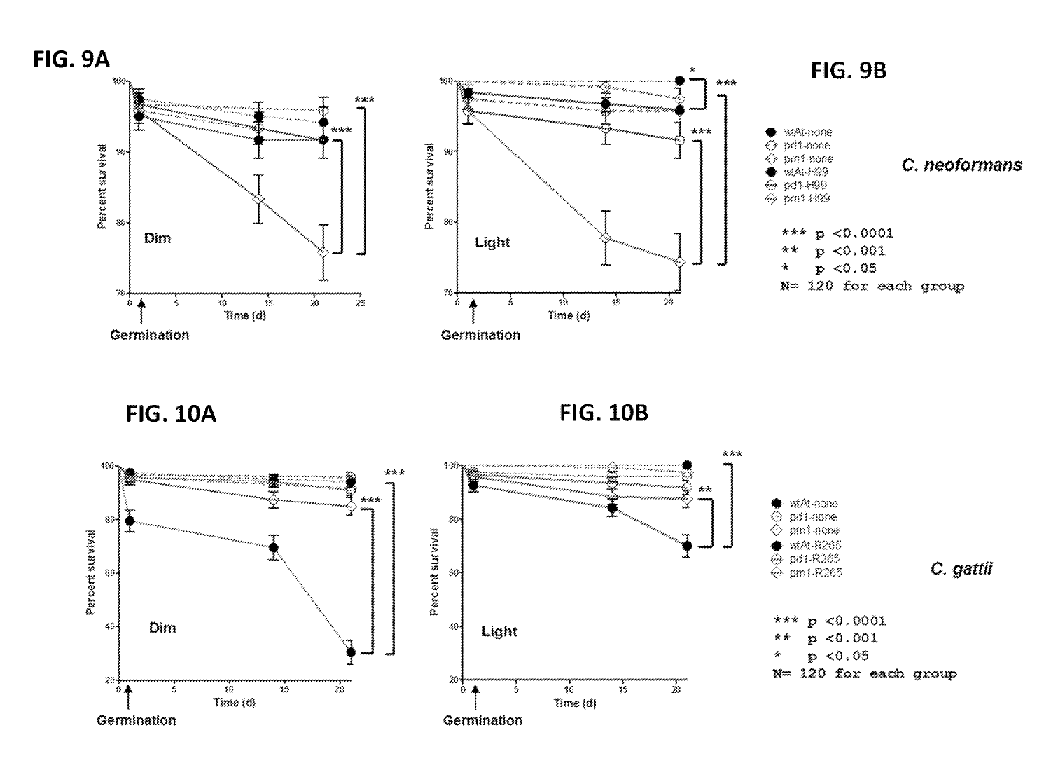

Inoculation of viable wild-type (wt) plant seeds with this strain resulted in reductions in seedling viability vs. untreated seedlings over the 21-day period in bright light (fungal treated: 95.8% survival vs. untreated: 100% survival; p<0.05; FIG. 9B), and a trend toward reductions in dim light (fungal treated: 91.6% survival vs. untreated: 94.2% survival; FIG. 9A), although the result in dim light did not reach statistical significance. Interestingly, the Atprn1Δ mutant (defective in pirin1; PRN1, prn1-) was markedly more susceptible to fungal inoculation than the wt At seedlings under both dim (fungal treated: 75.8% vs. untreated: 91.6% survival; p<0.001) and bright light (fungal treated: 75.8% vs. untreated: 97.5% survival; p<0.001), indicating a role for AtPRN1 in protection of seedlings from C. neoformans. However, mutation of a second effector of the Arabidopsis GPA1 signaling pathway, PD1/ADT3, that acts through the phenylalanine synthetic pathway to stimulate phenylpropanoid compounds (Warpeha et al., Plant Physiol, 140(3):844-55, 2006), resulted in a trend towards increased susceptibility that was not as severe as the Atprn1 mutant. This indicates a role for the PRN1-dependent G-protein-regulated stress pathway in defense against C. neoformans plant infections.

Co-Incubation of Cryptococcus gattii with Seeds from Arabidopsis thaliana Reduces Germination and Leads to Stem Lodging.

The ability of C. gattii to infect germinating seedlings was determined. C. gattii appears to be a more virulent pathogen in humans, affecting primarily immunocompetent individuals (Byrnes and Marr, Curr Infect Dis Rep, 13(3):256-61, 2011). The strain used was obtained from the recent Vancouver Island outbreak (Stephen et al., Can Vet J, 2002. 43(10):792-4). As shown in FIG. 9A, inoculation of C. gattii onto agar containing wt plant seeds under dim light resulted in greater plant virulence than C. neoformans, with both a reduction in germination (fungal treated group: 21 failures vs. untreated: 3 failures; p<0.001) as well as poor survival over the 21 day period (fungal treated: 25.8% vs. untreated: 94.2%; p<0.001). Seedlings were markedly less susceptible when exposed to an equivalent inoculum of C. gattii in bright light (light: 70.0% vs. dark: 25.8%, p<0.001), similar to the protective effect of blue light exposure reported in Arabidopsis for other plant pathogens such as the turnip crinkle virus (Jeong et al., Plant Signal Behav, 5(11):1504-9, 2010). Interestingly, the Atprn1Δ mutant (fungal treated: 84.2% survival vs. untreated: 91.7%) and the Atpd1/adt3Δ mutant seedlings (fungal treated: 90.8% survival vs. untreated: 95.8% survival) were more resistant to the effects of C. gattii inoculation compared to that of the wt seedlings (p<0.001). This indicates that C. gattii has evolved as a more effective plant pathogen than other strains of Cryptococcus, such as C. neoformans, and expands the ecological niche of the fungus to infection of live, germinating plants. In addition, greater virulence toward wt than GPA1-effector mutants indicates that this plant pathogen has evolved a unique mechanism to exploit the G-protein regulated stress pathway, normally important in resistance to pathogen stress.

Fungal Burden is Associated with Fungal Virulence in Arabidopsis thaliana Seedlings.

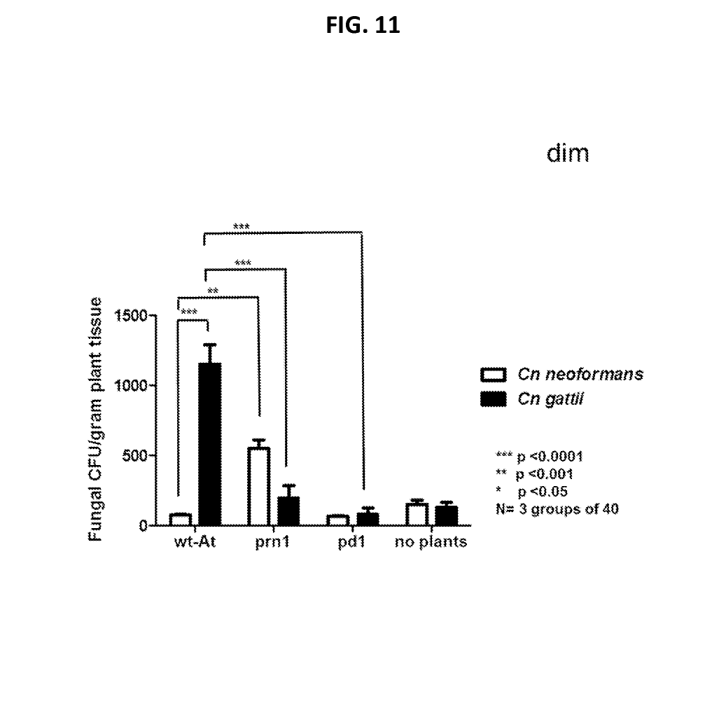

Whether plant stem lodging was associated with seedling fungal burdens was determined. At 14 days after inoculation, seedlings were harvested and analyzed for tissue fungal burden by culture after careful washing and tissue homogenation. As shown in FIG. 11, after infection of wt plant seeds, C. gattii showed increased fungal colony counts compared to the C. neoformans H99 strain (1152 vs. 75 CFU/g plant tissue; p<0.001), consistent with its greater pathogenicity towards plant seedlings exhibited in FIGS. 9A, 9B, 10A, and FIG. 10B. Colony counts of C. gattii were reduced in the Atprn1Δ and Atpd1/adt3Δ seedlings compared to wt seedlings (199 and 79.5 CFU/g plant tissue; p<0.001 in both), correlating with the reductions in killing observed for these fungal strain and seedling combinations. In contrast, for C. neoformans infections, the Atprn1Δ mutant seedlings showed increased fungal burden compared to wt seedlings (p<0.001), corresponding to increases in virulence against the mutant plant seedlings exhibited in FIGS. 9A and 9B. This implicates a role for plant-associated fungal proliferation in plant pathogenesis by two different species of Cryptococcus.

C. Neoformans and C. Gattii Lead to Tissue Invasion of Seedlings after Co-Inoculation of Fungal Cells and Seeds of Arabidopsis thaliana.

Since killing of seedlings within the 21-day time frame could be due to indirect effects of fungal products, we undertook a microscopic study to determine whether fungal invasion was present after inoculation. Seedlings were gently washed 14 days after inoculation, fixed, embedded and sectioned. While 14-day seedling invasion could be observed by microscopy in wet preparations, sectioning was performed to minimize the likelihood that tissue co-localization was the result of superimposition rather than fungal residence within plant tissue.

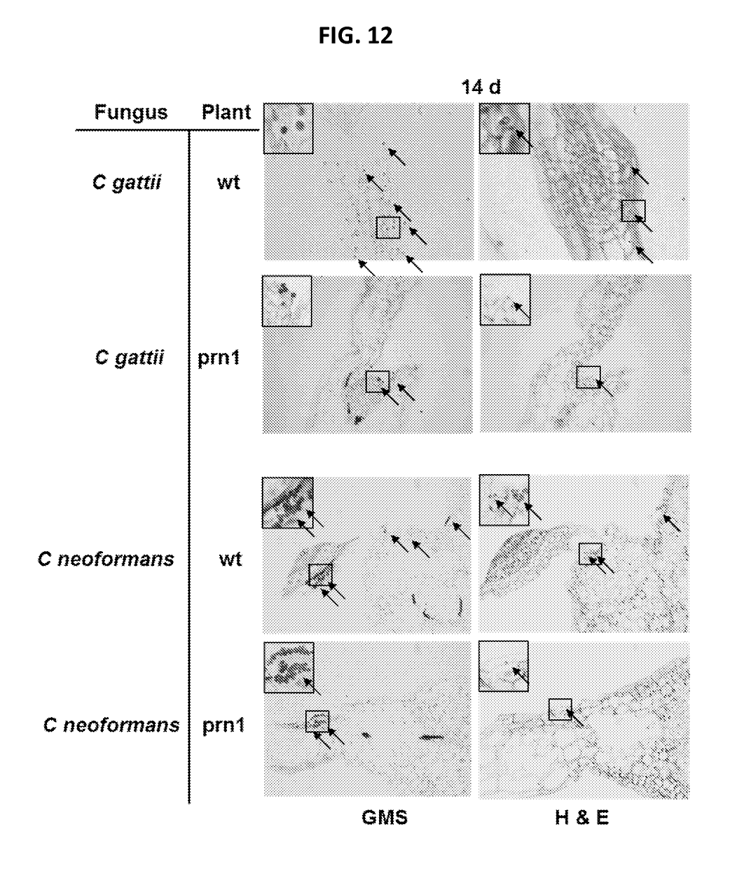

As shown in FIG. 12 (top panels), numerous C. gattii yeast cells (see arrows) were observed within plant tissue revealed by either Gomori methamine silver (GMS; left panels) or hematoxylin-eosin stain (H & E; right panel). Invasion of stems was also evident in the Atprn1Δ mutant seedlings, although to a lesser extent than that of the wt seedlings, consistent with a lesser degree of virulence against the mutant seedlings. In addition, as shown in the lower panels of FIG. 12, the C. neoformans strain displayed both colonization and tissue invasion. In the case of the Atprn1Δ mutant seedlings, C. neoformans was observed within plant tissue, further corroborating infectious killing of these seedlings by this fungal strain as shown in FIGS. 9A and 9B.

### Laccase Mutants of C. Neoformans Show Attenuated Virulence Towards Arabidopsis Seedlings.

To demonstrate the role for laccase processing of diphenolic compounds in the killing of Arabidopsis seedlings by C. neoformans, a lac1Δ mutant strain of the fungus was used to inoculate seedlings. Seedlings were grouped at a higher density to simulate germination from a point location, which increases susceptibility to plant pathogens (Syuuichi et al., J. General Plant Pathol., 76:370-376, 2010).

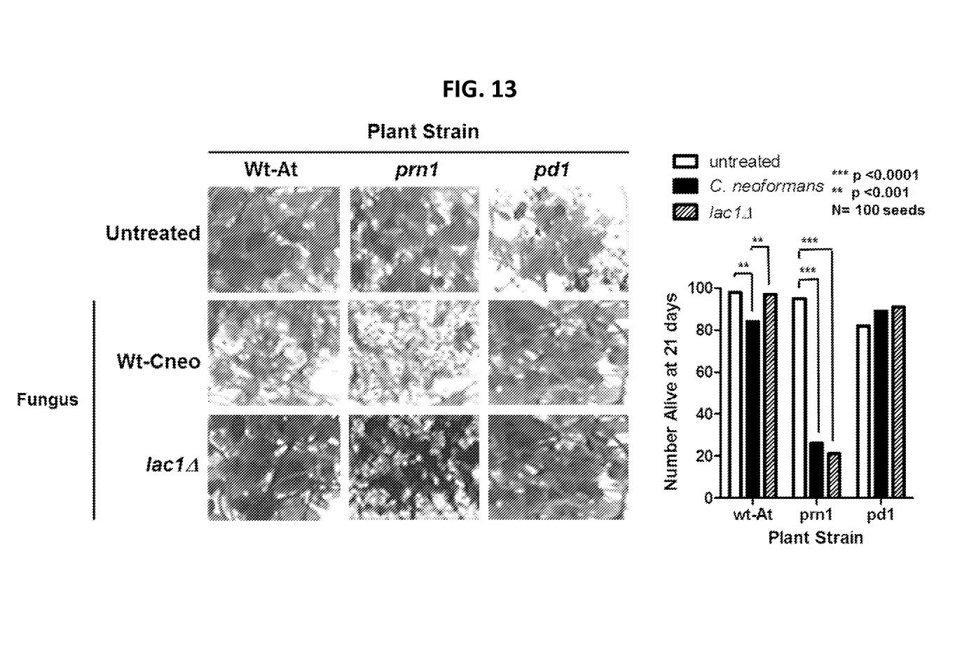

As shown in FIG. 13, inoculation with wt C. neoformans resulted in browning and death of seedlings at 21 days (untreated: 98 survived vs. fungal treated: 84 survived; p<0.01; N=100 seeds), whereas treatment with C. neoformans lac1Δ strains resulted in attenuation in virulence, with little changes in morphology as well as survival comparable to untreated seedlings (lac1Δ-treated: 97 survived vs. wt fungal treated; p<0.01; N=100 seeds). The surviving plants of these fungal infections switched over to reproductive mode (bolting, etc), albeit delayed compared to untreated plants, hence permitting coexistence of plant and fungus. These data indicate that oxidation of diphenolic compounds by fungal laccase plays a role in plant virulence as much as it plays a role during mammalian virulence.

Susceptibility of the Atprn1Δ mutant to the C. neoformans wt strain was again increased compared to the wt seedlings, and was increased somewhat compared to an equivalent fungal-plant challenge described in FIG. 10B, most likely due to increased plant density. However, inoculation of the Atprn1Δ seedlings with the C. neoformans lac1Δ strain did not attenuate virulence compared to the wt fungal strain as it had in the Arabidopsis wt seedlings, indicating that plant factors that require the laccase virulence factor for infection may be expressed in a PRN1-dependent manner.

### Discussion

Successful infection of intact germinating seedlings with the inoculation of Cryptococcus cells dispersed via a thin film across the top of minimal medium (non-enriched, no sugars or added organic matter), which contained ungerminated seeds less than 2 mm below the surface of medium under conditions simulating low and moderate daylight, is demonstrated. In contrast, for example, Springer et al. (PLoS One, 5(6):e10978, 2010) used Arabidopsis leaves of mature seedlings (4-6 leaves from each plant indicating a 3 week old, light-grown plant) that had been wounded and grown in a growth chamber, indicating full light conditions. Typically, older, light-grown plants with mature outer cuticles are resistant to fungi, invasion of which typically requires specialized invasive structures, such as the appressorium of the rice blast fungus Magnaporthe grisea (Liu et al., Eukaryot Cell, 6(6):997-1005, 2007). Seeds of plants have varying amounts of stored flavanoids to provide antioxidant materials to young growing seedlings until the chloroplasts are fully functional. In very dim lighting, such as that found in a temperate or tropical rainforest with only 2% sunlight reaching the forest floor, it takes longer to fully establish the photosynthetic apparatus and to develop the cuticle, suberinized roots and extra-cuticular structures that prevent pathogenic attachment and penetration into cells. This phenomenon is typical of the protective effect of light exposure reported in Arabidopsis against other plant pathogens, such as the turnip crinkle virus (Jeong et al., Plant Signal Behav, 2010. 5(11):1504-9). In addition, previous studies have shown that high concentrations of the fungus inhabit soils with high amounts of lignin, a product of the plant phenylpropanoid pathway, as reported for soils in the temperate rain forests of the Pacific Northwest (Hattenschwiler et al., New Phytologist, 2010. 189:950-965; Kidd et al., Eukaryot Cell, 2005. 4(10):1629-38). Growth in high lignin soils is facilitated by fungal laccase which breaks down lignin polymers (Kawai et al., Arch Biochem Biophys, 262:99-110, 1998). Thus, if fungal organisms persist in soils and tree surfaces due to high concentrations of lignin substrates, it is likely that plants acquire infection from contact of seeds in soil, where infection can ensue soon after germination. Infection is likely optimal at the forest floor with limited light, which delays development of seedlings and prevents optimal resistance to the fungus. In summary, young, light-deprived seedlings both contain rich nutrients and are most vulnerable to cryptococcal infection in the first few days post-germination. This indicates that removal of leaf litter in areas of high cryptococcal environmental contamination may serve to reduce infectious exposure to humans.

Interestingly, while C. neoformans was able to infect germinating seedlings, C. gattii was more successful as a plant pathogen, especially under conditions of low light. This indicates relative differences in plant pathogenicity of the two species and different infective strategies. For example, the finding that Arabidopsis mutants of the GPA1 pathway, Atprn1Δ and Atpd1/adt3Δ, were more resistant than wt plants to C. gattii inoculation was unusual and indicates that C. gattii evolved specific adaptive responses that anticipate and exploit plant host defenses induced by the G-protein stress response pathway. Light-independent infection by C. neoformans indicates an adaptation to plant defenses induced during light exposure and could suggest infection of seedlings heavily exposed to light. Notable is that, due to the integrity of its cell wall matrix, Cryptococcus neoformans is particularly resistant to photodynamic killing (Fuchs et al., Antimicrob Agents Chemother, 51(8):2929-36, 2007), indicating success by this species in high light environments. In contrast, facilitation of infection in dim light by C. gattii indicates adaptation to infection of plants found in the forest floor, such as that encountered in the extensive forests of the Pacific Northwest. These data show the diverse abilities of C neoformans and C. gattii to infect intact germinating seedlings.

The data provide insights into the intersecting roles of fungal laccase and plant flavonoids. The laccase enzyme is a major virulence factor of C. neoformans for mammalian infection (Salas et al., J Exp Med, 1996. 184(2):377-86), where its presence has been used as a marker of pathogenic species of Cryptococcus. Fowler et al. (Yeast, 2011. 28(3):181-8) reported that the commonly found plant flavonoids could act as a substrate for laccase, resulting in the formation of a defensive lignin-like cell wall coating. Such coated fungal cells have an increased resistance to cell death caused by oxidants produced by UV radiation and macrophages; thus, available plant flavonoids could confer a survival advantage to an invading fungus (Dadachova and Casadevall, Curr Opin Microbiol, 11(6):525-31, 2008). The Arabidopsis gene PD1/ADT3 is encoded by a prephenate dehydratrase/arogenate dehydratase gene and produces, in wt seedlings, phenylalanine and phenylpropanoids very early in development that could supply nutrients or lignin substrates to the fungus after processing by enzymes such as laccase (Williamson, J Bacteriol, 176(3):656-64, 1994). Interestingly, as shown in FIG. 13, deletion of PD1/ADT3 in Arabidopsis both increased susceptibility towards fungal infection and removed the advantage of the wt laccase gene, supporting an intersecting role for PD1/ADT3 and laccase. In addition, deletion of PRN1 of the Arabidopsis GPA1 signaling cascade led to increased susceptibility to C. neoformans infection and led to loss of a role for laccase in virulence, indicating a common feature between laccase and products of the plant GPA1 pathway. In addition to its role in utilization of flavonoids for synthesis of useful compounds, the armamentarium of laccase and peroxidase enzymes of Cryptococcus are also suitable for destruction of plant barriers to infection, such as lignin (Leonowicz et al., J Basic Microbiol., 2001. 41: p. 185-227).

Interestingly, the understory of Vancouver Island and the Pacific Northwest, where C. gattii is prevalent, is also fairly non-diverse, composed largely of moss and ferns, and, due to the low light and dampness, few angiosperms. Moss and ferns do not produce lignin but angiosperms, represented by species such as wild ginger and young seedling trees possess quercetin and other phenylproanoids which are potential carbon sources for C. gattii. Recent work has shown a troubling correlation between a loss of biodiversity and the emergence of infectious pathogens (Keesing et al., Nature, 2010. 468(7324):647-52). Thus, addition of C. gattii to the catalog of plant pathogens in these regions could have increased pathogen diversity in a way that negatively affected plant diversity, or alternatively, reduced biodiversity may have promoted the optimization of C. gattii as a plant pathogen that facilitated its emergence as a human pathogen. These results also extend the role of laccase as a virulence factor in plant pathogenesis. Cryptococcal laccase has also been shown to be required for protection from killing by free-living amoeba (Steenbergen et al., Proc Natl Acad Sci U S A., 2001. 98:15245-50), indicating the importance of this enzyme to a number of processes useful for environmental cryptococcal isolates.

### Example 4

### Content of prn− Extracts

prn1- extracts were generated as described in Example 1. Similar extracts were generated from wt plants. The extracts were analyzed using liquid chromatography and mass spectrometry. Agents detected that had a 5-fold or more difference between the prn1- and wt plants were as follows: quercetin, propionic acid derivative, carbonic anhydrase, piperidine naphthalene-2-carboximida derivative, theanine derivative, di(n-acetyl-d-glucosamine, prednicarbate, methylsalicyluric acid, a compound highly similar to 2S-hydroxy-10-undecanoic acid, a compound similar to aminobenzofurazan, and two compounds highly similar to specific antibiotics, and one compound that is highly similar to a carbonic anhydrase inhibitor.

It was observed that the following compounds were present in amounts at least 5-fold higher in prn1- extracts as compared to wt extracts: three quercetins, antibiotic-like derivative compounds, methylsalicyluric acid compound, highly similar to 2S-hydroxy-10-undecanoic acid, piperidine naphthalene-2-carboximida derivative, and a benzoic acid derivative. It was observed that the following compounds were present in amounts at least 5-fold lower in prn1- extracts as compared to wt extracts: inhibitor of glyoxalase, coelenterazine-like compound, pyrrolo-pyrazole derivative, sulfonamide inhibitor of carbonic acid, propionic acid derivative, prednicarbate, theanine derivative and di(n-acetyl-d-glucosamine.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

1. A method of making a plant extract comprising quercetin, comprising
homogenizing aerial portions of seedlings obtained from a transgenic plant, wherein the transgenic plant comprises an exogenous nucleic acid molecule that decreases pirin activity in the transgenic plant, thereby increasing an amount of quercetin in the transgenic plant, thereby generating a prn− homogenate; and
obtaining a supernatant from the prn− homogenate, thereby resulting in an extract comprising quercetin.

2. The method of claim 1, wherein the method further comprises:
growing seeds of the transgenic plant in darkness at −0° C. to 4° C. for 24 to 72 hours;
growing seeds of the transgenic plant in darkness at 15° C. to 25° C. for 4 to 8 days;
exposing the transgenic plant to one or more stressors, thereby generating an exposed transgenic plant;
growing the exposed transgenic plant in darkness at 15° C. to 25° C. for 6 to 24 hours; and
obtaining aerial portions of seedlings from the exposed transgenic plant.

3. The method of claim 1, wherein the method further comprises:
growing seeds of the transgenic plant in darkness at 4° C. for 48 hours;
growing seeds of the transgenic plant in darkness at 20° C. for 6 days;
exposing the transgenic plant to one or more stressors, thereby generating an exposed transgenic plant;
growing the exposed transgenic plant in darkness at 20° C. for 24 hours; and
obtaining aerial portions of seedlings from the exposed transgenic plant.

4. The method of claim 2, wherein the one or more stressors is UV radiation, cold, drought, heat, salt, or hormones or combinations thereof.

5. An extract made by the method of claim 1.

6. A method of making quercetin, comprising:
homogenizing aerial portions of seedlings from a transgenic plant, wherein the transgenic plant comprises an exogenous nucleic acid molecule that decreases pirin activity in the transgenic plant, thereby decreasing an amount of functional pirin protein in the transgenic plant and increasing an amount of quercetin in the plant, thereby generating an extract;
obtaining a supernatant from the prn− homogenate, thereby resulting in a prn− extract comprising quercetin; and
isolating the quercetin from the prn− extract.

7. Isolated quercetin made by the method of claim 6.

8. A method of increasing tolerance of a plant to a stressor, comprising
expressing an exogenous nucleic acid molecule that decreases pirin activity in the plant, thereby decreasing an amount of functional pirin protein in the plant and increasing tolerance of the plant to the stressor.

9. A method of increasing tolerance of a plant to a stressor, comprising
contacting the plant with the extract of claim 5, thereby increasing an amount of quercetin in the plant and increasing tolerance of the plant to the stressor.

10. The method of claim 8, wherein the stressor is UV radiation, cold, drought, heat, salt, hormones, fungi, bacteria, arthropods, worms, products of biotic organisms, or combinations thereof.

11. A method of treating a tumor cell, comprising:
contacting the tumor cell with a therapeutically effective amount of the extract of claim 5, thereby treating the tumor cell.

12. A method of preventing an infection in a mammalian subject or plant by a toxic fungus, comprising,
contacting the mammalian subject or plant with a therapeutically effective amount of the extract of claim 5, thereby preventing the fungal infection.

13. A method of preventing a toxic fungus from growing on a surface, comprising,
contacting a surface with a therapeutically effective amount of the extract of claim 5, thereby preventing a toxic fungus from growing on the surface.

14. The method of claim 13, wherein the surface is a plastic surface, soil, or a surface in a hospital or other health care facility.

15. The method of claim 12, wherein the toxic fungus is C. gatti.

16. A method of increasing anti-oxidant activity in a subject, comprising:
administering to the subject a therapeutically effective amount of the extract of claim 5, thereby increasing anti-oxidant activity in the subject.

17. A composition comprising:
a plastic material; and
the extract of claim 5, wherein the extract is present on a surface of the plastic material.

18. The composition of claim 17, wherein the plastic material is a consumable plastic material.

19. A method of making a plant extract depleted of quercetin, comprising
homogenizing aerial portions of seedlings from transgenic plant, wherein the transgenic plant comprises an exogenous nucleic acid molecule that increases pirin activity in the transgenic plant, thereby decreasing an amount of quercetin in the transgenic plant, thereby generating a prn+ homogenate; and
obtaining a supernatant from the prn+ homogenate, thereby resulting in an extract depleted of quercetin.

20. The method of claim 19, wherein the method further comprises:
growing seeds of the transgenic plant in darkness at −0° C. to 4° C. for 24 to 72 hours;
growing seeds of the transgenic plant in darkness at 15° C. to 25° C. for 4 to 8 days; and
obtaining aerial portions of seedlings from the exposed transgenic plant.

21. The method of claim 19, wherein the method further comprises:
growing seeds of the transgenic plant in darkness at 4° C. for 48 hours;
growing seeds of the transgenic plant in darkness at 20° C. for 5 to 7 days; and
obtaining aerial portions of seedlings from the exposed transgenic plant.

22. An extract made by the method of claim 19.

23. A method of preventing an infection in a mammalian subject or plant by a fungus, comprising,
contacting the mammalian subject or plant with a therapeutically effective amount of the extract of claim 22 thereby preventing the fungal infection.

24. A method of preventing a fungus from growing on a surface, comprising,
contacting a surface with a therapeutically effective amount of the extract of claim 22, thereby preventing a fungus from growing on the surface.

25. The method of claim 24, wherein the surface is a plastic surface, soil, or a surface in a hospital or other health care facility.

26. The method of claim 24, wherein the fungus is C. neoformans.


SEQUENCE LISTING (16 sequences)

SEQ ID NO: 1 - DNA, 1057 bp, Arabidopsis thaliana
  CDS (72)..(935)
gcttttcttcgtttatcatcaccatcaccatcatcatcatcatcatcatcgtcatcatcatcatcatcatcatgacgtatgagaacaatagtgtaccaagaattgtaatcaagaaagttctagcaaagcttgagaaggaaggtgaaggagctgtcgttagaaatggcatcacaaagattgaccagaagttattggacccgttcgtgttgctagttgaattttccttttcactctcagctggattcccagatcatcctcacagaggttttgaaagtgttacatacatgctacagggaggtatcattcacaaagatcctaaaggtcataaaggtacaattcaagccggagatgttcagtggatgacagcaggaagaggaatcattcattccgaatttccggaagaagaagtaaacaatggtttacagctttggatcaatctcccttccactgaaaaaatgactgaaccaaaatataaggaactatcgagtttagacattcctcgagcagaagaaaacggagttgaggtcaaagtcatagccggagattcaatgggaatcaaatctccagtctacacaagaacaccaacaatgttccttgactttaccctcaagccaggatctcaaacccaccaaaccgttccagaatcatggaccgctttcgcttacattatagaaggcgatgaaggtgttttcggttccttgaactcttccgcaatatcggcccaccatgttgttgtgtttgggccaggggatttagttagcgtgtggaacaagtcaacttcgaggtcattgaggtttttgttgattgcaggggaacctatcggcgagcctgtggttcagtgtggtccctttgtgatgaattcacaggcggagatcgatatggcttttgatgactatcagaatgctaagaacgggtttgaaatggccaagtgttagaggttacagtgaaaagagtttgtaaacaaaaaccattctgattattttggattttgttgtctttattccacattcaagatgagatcaatgttaggggctataatatcaaccatctctttcat

SEQ ID NO: 2 - protein, 287 aa, Arabidopsis thaliana
MTYENNSVPRIVIKKVLAKLEKEGEGAVVRNGITKIDQKLLDPFVLLVEFSFSLSAGFPDHPHRGFESVTYMLQGGIIHKDPKGHKGTIQAGDVQWMTAGRGIIHSEFPEEEVNNGLQLWINLPSTEKMTEPKYKELSSLDIPRAEENGVEVKVIAGDSMGIKSPVYTRTPTMFLDFTLKPGSQTHQTVPESWTAFAYIIEGDEGVFGSLNSSAISAHHVVVFGPGDLVSVWNKSTSRSLRFLLIAGEPIGEPVVQCGPFVMNSQAEIDMAFDDYQNAKNGFEMAKC

SEQ ID NO: 3 - DNA, 1576 bp, Homo sapiens
  CDS (486)..(1358)
agttaaaaacagatttcccacaagaccgaccggagcgccgatcagagcacctgcccgggccacacatttcctcctggagcacagcaagtgccgcctaaattacccgagtgagcatctcttcccggcacgagaggcagggaggccaaagggccgccaagctggcctgggagaggcgtagggcggagcgagagtggagtgacattcccgagggcggagccccagggcctccgagacccgtagactcccgcctcccgcctcctctaggccgccggccgcgaagcgctgagtcacggtgaggctactggacccacactctcttaacctgccctccctgcactcgctcccggcggctcttcgcgtcacccccgccgctaaggctccaggtgccgctaccgcagcgtgagtacctggggctcctgcaggggtccactagccctccatcctctacagctcagcatcagaacactctctttttagactccgatatggggtcctccaagaaagttactctctcagtgctcagccgggagcagtcggaaggggttggagcgagggtccggagaagcattggcagacccgagttaaaaaatctggatccgtttttactgtttgatgaatttaaaggaggtagaccaggaggatttcctgatcatccacatcgaggttttgaaacagtatcctacctcctggaagggggcagcatggcccatgaagacttctgtggacacactggtaaaatgaacccaggagatttgcagtggatgactgcgggccggggcattctgcacgctgagatgccttgctcagaggagccagcccatggcctacaactgtgggttaatttgaggagctcagagaagatggtggagcctcagtaccaggaactgaaaagtgaagaaatccctaaacccagtaaggatggtgtgacagttgctgtcatttctggagaagccctgggaataaagtccaaggtttacactcgcacaccaaccttatatttggacttcaaattggacccaggagccaaacattcccaacctatccctaaagggtggacaagcttcatttacacgatatctggagatgtgtatattgggcccgatgatgcacaacaaaaaatagaacctcatcacacagcagtgcttggagaaggtgacagtgtccaggtggagaacaaggatcccaagagaagccactttgtcttaattgctggggagccattaagagaaccagttatccaacatggtccatttgtgatgaacaccaatgaagagatttctcaagctattcttgatttcagaaacgcaaaaaatgggtttgaaagggccaaaacctggaaatcaaagattgggaactagtggaaagcggaagagcaggtcttgatgtgtcctagaattttgccatttctgagattgagccattgaaggcattccatttctaaagcttatttagccggtgcttctaaagaattccacactaacgtgataacatggtttttgtaacaataaatgtaggatatttcctggcacatgcaaataaacctaatcattgtttctttaaaaaaaaaaaaaaaaaa

SEQ ID NO: 4 - protein, 290 aa, Homo sapiens
MGSSKKVTLSVLSREQSEGVGARVRRSIGRPELKNLDPFLLFDEFKGGRPGGFPDHPHRGFETVSYLLEGGSMAHEDFCGHTGKMNPGDLQWMTAGRGILHAEMPCSEEPAHGLQLWVNLRSSEKMVEPQYQELKSEEIPKPSKDGVTVAVISGEALGIKSKVYTRTPTLYLDFKLDPGAKHSQPIPKGWTSFIYTISGDVYIGPDDAQQKIEPHHTAVLGEGDSVQVENKDPKRSHFVLIAGEPLREPVIQHGPFVMNTNEEISQAILDFRNAKNGFERAKTWKSKIGN

SEQ ID NO: 5 - protein, 290 aa, Mus musculus
MASSKKVTLSVLSREQSEGVGARVRRSIGRPELKNLDPFLLFDEFKGGKPGGFPDHPHRGFETVSYLLEGGSMAHEDFCGHVGKMNPGDLQWMTAGRGILHAEMPCSEEPAHGLQLWVNLRRSEKMVAPQYQELKSEEIPKPTKDGVTVAVISGEALGIKSKVYTRTPTLYLDFKLDQGAKHSQPIPKGWTSFIYTISGDVYIGPDDAQQKIEPHHTAVLGEGDAVQLENKDPKRSHFVLIAGEPLREPVVQHGPFVMNTNEEISQAILDFRNAKNGFEGARTWKSKIGN

SEQ ID NO: 6 - protein, 313 aa, Dictyostelium discoideum
MLMNNYKSSDIKMNERVIGKISTSINTTDGEGVKIKRSIGSGEIGIRNDELSPFLLLDEIRSNESSDYMSGFPTHPHRGFITVTYMLKGEMRHNDNRGNQGLLKEGSAQFMVAGRGIVHSEMPIRNHEDQSFFAFQLWINLPSAKKMIDPSYQDYHSTEIPTIVSMDDTHFNNYTVKILAGQFQDTIGPIVDENLKTFFFDIELKPNTKFTDIIIPSTHNTFVYVYNGNGRFGGPVSRSKLVKINQVALFLNNGGDENSLDTIQVEAGSDGVKFLLLAALPIHNEKVVQYGPFVMNSDAEIKKAILDFRTNNF

SEQ ID NO: 7 - protein, 250 aa, Alicyclobacillus acidocaldarius
MAWMKFPGAHRAPIMFYEHARIFPSRTTPYIDPFLLLDHFSIQHPDGFPDHPHRGFEIITYVLRGAVAHADSAGHQSVIPEGGAQHVTAGRGIVHSEMPGTDGIDSGLQLWINIPRSDKGMDPGYEDIPPEALPVDEPAQGVRRKWIVGGGSPLKTHRPMTYQDVEMAAGASYTLKAPPYHQGFIFVLDGAGHLGEEEIPMQKGDLFIWHRAADEAFVPTPVRAAESLRAVMVFGEPVGERPIFNGPFVD

SEQ ID NO: 8 - protein, 325 aa, Streptomyces coelicolor
MPAVTVENPLTLPRVSAPADAVARPVLTVTTAPSGFEGEGFPVRRAFAGINYRHLDPFIMMDQMGEVEYAPGEPKGTPWHPHRGFETVTYIVDGIFDHQDSNGGGGTITNGDTQWMTAGSGLLHIEAPPEQLVMSGGLFHGLQLWVNLPAKDKMMAPRYQDIRSGSVQLLTSPDGGALLRVIAGELDGHDGPGITHTPITMVHATLAPGAEVTLPWREDFNGLAYVMAGRGSVGAERRPVHLGQTAVFGAGGSLTVRADEKQDAHTPDLEVVLLGGRPIREPMAHYGPFVMNTKDELMQAFEDFQKGRLGTVPAVHGMSGEGPGA

SEQ ID NO: 9 - protein, 231 aa, Escherichia coli
MIYLRKANERGHANHGWLDSWHTFSFANYYDPNFMGFSALRVINDDVIEAGQGFGTHPHKDMEILTYVLEGTVEHQDSMGNKEQVPAGEFQIMSAGTGIRHSEYNPSSTERLHLYQIWIMPEENGITPRYEQRRFDAVQGKQLVLSPDARDGSLKVHQDMELYRWALLKDEQSVHQIAAERRVWIQVVKGNVTINGVKASTSDGLAIWDEQAISIHADSDSEVLLFDLPPV

SEQ ID NO: 10 - protein, 291 aa, Rattus norvegicus
MASSKKVTLSVLSREQSEGVGARVRRSIGGPELKMLDPFLLFDEFKGGRPGGFPDHPHRGFETVSYLLEGGSMAHEDFCGHVGKMNPGDLQWMTAGRGILHAEMPCSEEPAHGLQLWVNLKRSEKMVEPQYQELKSEEIPKPSKDGVTVAVISGEALGIKSKVYTRTPTLYLDFKLDQGAKHSQPIPKGWTSFIYTISGDVYIGPDDAQQKIEPHRTAVLGEGDTVQLENKDPKRSHFVLIAGEPLREPVVQHGPFVMNTNEEISEAILDFRNAKNGFEGAKTWKSKIGNQ

SEQ ID NO: 11 - DNA, 1347 bp, Zea mays
  CDS (160)..(1047)
aacaacacaccccaccgccgccgaacaagtacatcaactactactcatatcacccaaaagcagttcctgctcctcgtgatcctcctgctgctcctcctcctcttctccgtcgtcttcttgatccccaccgtctcgttgcccccagctcggcctcggaccatgtcgtcctccgcctccgatgccgcgccgttcgagaagcccagggccgtggtcaagaaggtcctcgcggagtcgcagccagaggggcagggcgccaccgtccgcaggagcatcggcaggcatgagctccgcaacctggaccccttcctcctgctcgacgagttcacagtctccaagcccgccgggttccccgaccacccccacagaggattcgagaccgtcacctacatgctcgagggggccttcacccaccaggacttcgctgggcacaagggcaccatcaggacaggggacgtgcagtggatgacggcggggcgaggcatcgtgcactcggagatgccggcgggggacggcgtgcacaagggcctgcagctctggatcaacctctcctccaaggacaagatgatcgagccgcggtaccaggagctggagagcaaggacatcagccgcggggagagcgaggacggcggcgtggaggcccgcgtcatcgccggggaggccctcggcgcggcgtcccccgtgtacacgcgcacccccaccatgtacgtggacttcaccatgcgccccgggtctcacctgcaccagcccgtcccggagggctggaacgccttcgtctacgtcgtcgacggcgagggcgtgttcgggcgggagacggccacggcgcactactgcctcgtcctcggccccggcgacggcgtcagcgtgtggaacaggtccaccaggccgctgaggttcgtcctcgtcgccgggcagccgctcggcgagcccgtggtgcagcacgggcccttcgtcatgaactcgcgcgcccagattcagaaggccatggaagactactactatggcaagaacggcttcgagagggccggacagtggagctcctccgcctgatcggcgaggcggggagccgatggcgccaaattaatctcccgtgcaaactaaatatactttattaaatttgttagggtgctgagtttgactattttttagcaactacacttgtattggtagtatgtcatctagtatggcgaattggatattgtaataaacagtaaggaaaattgatagtctcaataaaccattgtatttgtagcatatggaaaattgtatatcaaggccatttgtgggtgattgctgttttccagtctaaagtatcagttatttatgcgtggagacaaaaaaaaaaaaaaa

SEQ ID NO: 12 - protein, 295 aa, Zea mays
MSSSASDAAPFEKPRAVVKKVLAESQPEGQGATVRRSIGRHELRNLDPFLLLDEFTVSKPAGFPDHPHRGFETVTYMLEGAFTHQDFAGHKGTIRTGDVQWMTAGRGIVHSEMPAGDGVHKGLQLWINLSSKDKMIEPRYQELESKDISRGESEDGGVEARVIAGEALGAASPVYTRTPTMYVDFTMRPGSHLHQPVPEGWNAFVYVVDGEGVFGRETATAHYCLVLGPGDGVSVWNRSTRPLRFVLVAGQPLGEPVVQHGPFVMNSRAQIQKAMEDYYYGKNGFERAGQWSSSA

SEQ ID NO: 13 - DNA, 891 bp, Ricinus communis
  CDS (1)..(891)
atgtctgcttctgatgatcagtcctctgccttcagtagaccaagaatggtcaccaagaaagtccttgccaagcctcaacatgagggtgatggtgctgttgttaggagaggcattggatgtaatgagctgaggttcttggatccttttctcatgctggatgatttttcagtgagtcctcctgctggttttcctgatcatccacatagaggttttgagactgttacatacatgcttcagggagccatcacccatcaagattttgcagggcataagggtacaattcatactggggatgtgcagtggatgacagcaggaagagggataatccactcagaaatgccagcaggagaaggtgcacaaaagggcttacaactttggataaatttatcttctgaagacaaaatgattgaaccaaggtaccaggaactactaagcgaggacataaaatgtgcagaaaaagatggggttgaagtgcgaatcatagccggagaatcaatgggagtaagatcaccagtttacaccagaacacctacaatgtacttggatttcaccctaaagcccagaacccagatgcatcaaagcattccagaatcatggaatgcatttgtttatatcattgaaggtgaaggagcctttggcatcagaaacacttcagtagtgcaagcttaccatgtcctagttttgagctctggagatggtctaagcgtatggaacaagtcctcttccaagacattaagattcgtgctagttgcagggcagcccattaatgaaccagtggttcagcatggcccttttgtgatgaacacacagaaagaaattgagcagaccattgaagattaccattatgccaaaaatgggttcgagatgggcaagtactgggcatctcagtaa

SEQ ID NO: 14 - protein, 296 aa, Ricinus communis
MSASDDQSSAFSRPRMVTKKVLAKPQHEGDGAVVRRGIGCNELRFLDPFLMLDDFSVSPPAGFPDHPHRGFETVTYMLQGAITHQDFAGHKGTIHTGDVQWMTAGRGIIHSEMPAGEGAQKGLQLWINLSSEDKMIEPRYQELLSEDIKCAEKDGVEVRIIAGESMGVRSPVYTRTPTMYLDFTLKPRTQMHQSIPESWNAFVYIIEGEGAFGIRNTSVVQAYHVLVLSSGDGLSVWNKSSSKTLRFVLVAGQPINEPVVQHGPFVMNTQKEIEQTIEDYHYAKNGFEMGKYWASQ

SEQ ID NO: 15 - DNA, 912 bp, Carica papaya
  CDS (1)..(912)
atgcctgagggagagaattcttctgtgggagttcgtgaacccagattagtcgtgagaaaattcttggctagacagcagcatgaaggagttggagccattgtcagaagaagcataggaaggtttgagctgagatactttgatccttttctggttctggatgaattttcagtaactgctcctgctggatttcctgatcatccacacagagggtttgaaacagtcacctacatgttacagggagctgttacacatgaagattttgaagggcataaaggaaccataggagctggagatctgcaatggatgactgcaggaagaggaattgttcattcagaaatgcctgcaagtcagggaacccaaaagggtttgcaattgtggatcaatctcccctcaaagtacaaaatgattgaaccaagatatcaagaaatgttaagtaaagatattgtaaaagttacaagagacggaattagtgttagggtaatagcaggagaagccctaggggctaagtcactaatctatactagaacaccaaccatgtatttggatttcactctcgacccaggagctaagctccgacaaccaataccaagatcatggaatgcctttgtttatgtcttagaaggcgatggtgtgtttggcaactcaaaatcttcgtctgtttcagctcatcaccttttattattaggatctgggaatatgttgaaggtatggaacaaatcaacatctaaacccgtgagatttattttggttgggggtgagccattgggtgagccaatagtgcaatttggtccatttgtgatgaacacacaggaagaaattgatcagactattgaagattttgagaattttactaatggatttgagaaagcaagacaatggagatctcaagctgcactccgcttagatttttag

SEQ ID NO: 16 - protein, 303 aa, Carica papaya
MPEGENSSVGVREPRLVVRKFLARQQHEGVGAIVRRSIGRFELRYFDPFLVLDEFSVTAPAGFPDHPHRGFETVTYMLQGAVTHEDFEGHKGTIGAGDLQWMTAGRGIVHSEMPASQGTQKGLQLWINLPSKYKMIEPRYQEMLSKDIVKVTRDGISVRVIAGEALGAKSLIYTRTPTMYLDFTLDPGAKLRQPIPRSWNAFVYVLEGDGVFGNSKSSSVSAHHLLLLGSGNMLKVWNKSTSKPVRFILVGGEPLGEPIVQFGPFVMNTQEEIDQTIEDFENFTNGFEKARQWRSQAALRLDF